SEQUENCE LISTING (41 sequences)

SEQ ID NO: 1 - DNA, 12657 bp, Danio rerio
atggcgcacagctgtcggtggcggttccctgctcggcccggagggagcagcagctcgggcaccgggaggaaagcgggccgaattcgagtcaatgcttccctccttatcagcgcgggaacaaatccgaacgcgaacgggctcgggcccggtttcgacgctgcgttgcaagtgtccgctgccatcggcagcaacctgcagaaatttcgggatgttttgggggagtcgagcggctccagtagcggggaggaggaatttggaggctttaccacagttagtgacaacagaagactacatagccctggaagaacgtcaataggttctatcacaccagacaagaagcccagaggacgtcctcctaggactcctgctgtgcagagggttggcactgatgctgaaacagcccctttgcctgttgccacatcacctacagagaagttaaagcgacaaccagggaggcctcctggcactagagaaaaaaaaagaggtcgccctcctgcttctgtcagccaaaggacctggcaacacagcggccatgcactgcctgaagagggtagagaagtcccacaggagtgcagctccagtcctgtacacagcaaggaaggtgtggaggaaaacaaggagaaaaggcaaactccacttgggtctgggcaccatcagggatctgaggctaagcttcacaaagtcagtcgggagtccaaggtgaccaaactgaaaagactgcgggaagtcaaactgagcccactgaagtctaagctgaaggccattgtaaggaaaacagtcactgttcctggtaagcaaagacggaagcgaggcagaccaccttctgcagagcgcctcaaagctgaggctgctgctgctgccgctgctgctcaagctgcaaatgcatccatggcccaggagacatccacaacagcaccgaggactgctaagaaaaaggcctttagggttcgtcggtcacaagacttgggtgctcgcactccacatgagctcagggcttctcatactgctgatgaacacactcactcagattcccaagactcccctacagcagctgacccattgaccccaactaaagttggcaggcctttggggttacgtcagagccctcgccatatcaaacctgtgcgggttgtccctccctctaaacgcactgatgccacaattgcgaagcagctattacagagggccaaaaagggggcccagaagaaaaaactgttggaaaaagattctgttggcacacaaggaaaagccggtcttgaggctgggaagcacagaaggcgaacacaattaaccaacattaggcagtttatcatgcctgttgtgagcacagtgtccttgcgcatcatcaagactccaaagcgatttattgaagatgagggcagtttcagcactccaccaccacacatgaaaattgctcgattagaatcagcactgactgccccagcaccccaacccgcaacaccatccaccccagctctcgtttccacagctccttctacaagtggaactactgccacacctgggtcagggtctgcggttgagtctctccctcctccaccacctcctgtctcaacgggcagcactactgccattgcagctagtctccttaacagcagctgcaacaatagcactagcaatggacgcttcagtagcagtgcggcatcctgtggctccagtgctgtttcgcagcattcctctcagctctcttctggtgagccatctcgctctactagccctagccttgatgactcctcctgtgattcccaagcctctgagggtacgcaggccctctcagaagaggttgatcattccccagcctctcaaggagagacagaggccagtttgcaccatgcctctcacccaccatcaccaacatctgagccagagccagaccatatagttttggagcacagcaggcggggtcgcagaggtcagagtcatagacgaggtgctgtagtggcacgtggtaggggcaacctaattattggaagaaaacaggctattatcagcccagccacaggagtttcacaagctggatctcaacaggcctcttccactgcatcgtcttcatcttccccaccgccgcctcctcttcttagtcctcctcagcctcctcagacagcttcatccaatgcagcagaacatcactcccattcaccttggatgatgtcacactccattgccccctttctacctacttcttcaatactctccagttctcatgacaaacgtcgctcaatactacgagagcctactttccgctggacatctttgtcatgtgctgaaaataaatacttctcctctgccaagtatgcaaaggagggtcttatccgcaaacccacttttgacaactttcggcctcctccactgactgctgaggatgtgggactcatgcctccagtcactggtggaggtggtgtcacttcaggaggcttcccagcacctggtggtgcggctgggacaggtacaagacttttctcccccttgcatcatcatcctcaccacaatcaccaccaacattcctcatcacgttttgaaacaccactccaaaagcgcacccctttactacgccctcctttctttactcccagtccagctcattcccgcatctttgagtcagtaaccctcccatcctcctcagggagcagccctggatctctgtctcccctacaagtctcaccaacatcaagcaaaaagaaaaaggggtcaaggttccctcgtgggcaaccccggtcaccttcacactctatgattaccaggagctgtcagtcaggcgttccaacagggaagtcttctgaacaatccattattagcagttctgtccctataaccgtaactggaaattctagtccattacctggggttgcagtcagtccacttgctgccagtgccttaactcaagcatctttcagtggcttcccctcaggctccattggtcttacaagccatggagtttcagatgggcggcgagcagcagggggtctcagtgtaagtgggaattctgcttcatcttcacagctcttccctctttttacaccaagtcctcaagcatcaggtgggggtactggaaaagcaggaaaggaacgcggtatatctgctaccagagacacaggtacaaaggagaaggaccgggagatggagaagagcagggaacgtgaaaaggaaaacaaaagagatggaagaagagattgggataaaagagggaaaagccttccatcagaagcttcccccagttctatatctagtttctttggtttggaggctattgaggaatctctcacccaaaaaaggacccctggccgaaaaaagtcagtcacagttgactgtgcagaggcctctccaagtgactctgcagcagttcaggctgttgggtccttgtcatcaaagggtcggctgactaaaaaaggcagacctccagagaagagtattgaatcagagggagtagagagggaaaaggacaaagagaaactgagtgcccttacccaagcaggtcagatggggaaacccccaacaactacatccatagactccatattagatcgtgccgagaagcggcctgttacagacagacgtgttgttagactgctgaaaaaagctaaagcccagctcaataagatagagaagcgagagttacaacctggtgatcaacccaaattgccgggacaagaaagtgactcctctgagacttcagtccgtggtccacgaattaagcatgtgtgtcgtcgtgcagctgtagctctgggtcgcaaccgtgccgtgttcccagatgatatgcctacccttagtgccttgccatgggaggagagagaaaagatcttgtcttccatgggaaatgatgacaaatcatcagtggcgggatcagaggaggcagagccacccacacctcctattaagccagtgacaaggcagaagacagtccatgaggccccacccagaaagggccggcgctctcgacgctgcgggcagtgtccaggctgccaagtcccaaatgactgtggggtgtgcaccaactgcctggataagcccaaatttggggggcgcaatattaaaaaacaatgttgcaaagtacggaagtgtcagaatttgcagtggatgccatcaaagtttcttcagaagcaagcaaaaggtaaaaaagacaggaggaggaataaattgtctgaaaaaaaggaattgcaccacaaatcccagtgttctgaagcaagccccaagtcggttcctcctccaaaggatgaacctccccgtaagaaaagtgaaactccgccaccagcacagggagatgacaaacaaaagcagacacaaccttcatcaccatcctcaccagcttcctccccaaaggaccctctcctttctagtcctcctgacgaccacaagcattcactgacttctctgagctcggcttgtagaaaggaacggaagcagcagcccagttcttcacccaccttcctgcatgctgccccatcttccccaccagcacagtcccagcattccttgcagcagccatgccaaatgccagcaaaaaaggaaggtcttacaaagtcgcagtcgcacactgagcccaagaagaaatctcaacaacaaagtcaacccagctctgccacagacacagcacctgatgcaaagctaaagaaacagaccactcgatgcgttcaaccactcaagcctaaaccaaaagaaaaggagaaacagctacccaaacctgacagcagtactttaaactcccagagcactccttcgactgggggcacggccaagcagaaagcgccctacgatggagtgcatcgaatcagagtggatttcaaggaggactataacattgagaatgtgtgggagatgggtgggctcagcattctcacctctgtgccaatcaccccacgggtggtgtgctttctttgtgccagcagcggtaatgtagagtttgttttctgccaggtgtgctgtgaacccttccatctcttctgcttgggggaggcagagcggcctcatgacgaacagtgggaaaactggtgttgccgccgatgccgcttttgccatgtttgtgggcggaaatatcagaaaaccaaacagctactggagtgtgacaagtgccgaaacagctatcaccccgagtgcctaggacccaaccatcctaccagacccaccaagaagaagagagtctgggtttgcaccaagtgtgtgcgctgtaagagttgtggagccaccaaaccaggaaaggcctgggatgcccagtggtcacatgatttctctttgtgtcatgactgtgccaaacgtttaactaagggcaacttgtgcccactttgtaataagggctatgatgacgatgactgtgacagcaaaatgatgaagtgcaaaaagtgtgaccgctgggtccatgccaaatgtgaaagcttaacagatgacatgtgcgagctcatgtctagcctgcctgagaacgtggtctacacttgcacaaactgcactgggtcccatcctgctgagtggcgcactgtcctagagaaagaaattcagaggtccatgcggcaagttctcaccgccttgttcaactcccgcacatccacacacctgctccgctatagacaggctgttatgaagccacctgagctcaacccagagaccgaagaaagccttccctcacgacgttccccagagggtcctgatccccctgtgttaacggaggtctctccaccaaacgattcgccgctcgatttagagtctgtggagaagaaaatggattctggatgctataaatctgtgctggagttcagtgatgacattgtgaaaatcatccagacagccttcaactcagatggaggtcagctagagagcaggaaagccaacagcatgctcaagtccttttttattcggcaaatggagcggatttttccatggtacaaggtgaaggagtccaaattttgggagacaagcaaagcttcttccaatagtggactgcttcccaatgttgttctgccaccttctttggatcacaactatgcccagtgccaggagagagaggagatggccaaggctgggcagtccgtgcacatgaaaaagatcatcccggctcctcatcctaaagcccctggagaacccaactctctgatggcccctacaccaccccctcctccaccaatgcttatccatgaccacagccttgagggcagccctgttgttcctcctcctcctggtgttggtgacaacagacagtgtgcgctttgtctgaattatggagatgagaaaacaaatgattgtggcagactgctgtatattggtcataatgagtgggcgcatgtgaactgtgccctgtggtcagcagaggtgtatgaggatgttgatggagctctgaaaaatgttcatatggctgtaagccgaggcaaacagctgcaatgtaagaattgtcacaaacctggagccactgtgagttgttgtatgacctcctgcaccaacaactaccatttcatgtatgcacgtcagcagcagtgtgcctttttagaggacaagaaggtttactgtcagcatcacaaagatcttgtaaagggcgaggtggtgccagagtctagttttgaagtaactcggagggttcttgtggactttgagggaatccgtctgagaagaaaatttgttaatggacttgaaccagataacatacacatggtgataggatctatgaccatcgactgtctaggtatgctaactgaactgtcagactgtgagaggaagctatttcctgtgggataccaatgttcaagggtctactggagcactctagatgcccgcaagcgatgtgtttataaatgtagaatattagtgtgcaggcctcctttgagtgaaactttgaataagaacatagcagctcaagaggagaaccacacagttatccatagtcctccacctgtttcagtggatacctttttgcctggacccatagattccacaaaaccatcaaatgtgccttccacaccaaaaccacgagtttattttaggaacaggcaccccagctttccaccatgccatcgttctccttcaaccagaccacttccctcaccagatggttttaataatacaggccatgagattgtgactgttggagaccctctgctgagctctagtcttcgaagcattggatctcgtcgacacagcacttcctccatctctgctcaacaacctaggcaaaaggtttcctcccctccacagggaggtacagtatacagccaaacaggcaattcatctgcctctttcatgtcttcaacctctaaagaacctttaaccaaggacacagataaaggaagggtatcatctggagagacatctttcagtcgagaaccaaattcgataaacattggagcacagcgtcgactcagttttggtttcactgaaagagtggatggtagtaaagaagcaaccaaaaagcactctgatggtgagagtttgaggtcatcgcaaccagctagtgtaagtcaggtgtctccacctcttggaactgcagtattgacaggacatcagagagcaagtggtggtataaaaaatgagaaagggaaacaagcaacaaaagataatgacctgccagctggagccacttttatgtccagtcatccacttgccatgcttccaaaagacaaagctaatccaaacaaggaaggaaatatgacatcaatggctgcattaaaagacacagtaaagacaggttctccgcaaaggatttacaataaaagtgggagtaggaagtctcatgactatgcgtcaggcccagctgcagtggtagcaatgaaacctctctggtcatctggtgccaagttgggagaggaagacataaagcgtggctttcaggcaagtgctggtatcactggtagtcacgggacctctagcaccaaagaaaaacactccaaagtcaaaatgaatgtcagcagggatgtttcaaaagaaagaaaagagactcctcaaaaccgaaatgcggtgctcaacagcaactctaaaagcagcaatgtcaaaacacaaggtcaggttccaccacctcacaacatcagcaataaagccacagcactaagcagcaacacagggtctggcactgtggaagttaataaatttgatcagaaggaagtggagaagccattaaagtccaaagagagatttagctttgagaaaaagcatacttcagccatggatgctattcaaccgaaagcagggtcagagagaagtattcgaccaccacaagtgcaccctaagtcaagtaaggaagttcctctagtgggaaagaaacacaccgaaaggctttctttaatgtctcagaaaatggatcctaatcgaacaaaagcagtcagcatatcacctaacacacaaacatacacttctgttacccctagcaaccagggcccccaaagaaggtcgtctcgagctatggttttctccccatctgcaagttcagagagctctgaatcagacagccacatccacccggatgattctgaagagcatctcatggaccaccagtgtgctgatgatggggaggacaataatttagaggatgaaggcagtgtcgataaacaccacgaggaggatagtgatggttcagcaggttcagcaaaacgcagatacccaaggaggagtgcccgtgctcgatctaacatgttttttgggttaactccattctatggtgttcgatcgtatggtgaggaagacatacccttttacagaagtggtgaaatctctatgaagaagcggactgggagcagcaagcgctcagccgaagggcaggttgatggagcagatgatatgagcacatcttcttcagcagacagcggagaggatgaagaaggaggaattggctccaataaggatacttactattacaacttcacacgcactatgataaaccctagctctggtcttccatctattgctggtattgatcagtgtttgggaagaggttcacagatccacagattcttgagggaccaggcaaaggagcatgaagatgacagtgatgaagtttcaacagcaaccaaaaacttggagctgcaacaaattggtcagctggatggtgtagatgatggttcagagagtgacattagtataagtaccagtagcacaaccactgctactacttcatccacacaaaaaggttcaacaaaaaggaaaggtagagaaagtaggactgaaaaatcaaatgttgactcagggaaggaggcagtaaataccactagtaacagccgtgacagtcgaaaaaatcaaaaggataactgtcttccattaggaagtgcgaaaacacaaggacaagacccacttgaaactcaattatcactcaccacagatctgctcaagtctgactctgataacaacaacagtgatgactgtggtaacatcttaccctctgatattatggagtttgtgctcaataccccttcaatgcaggctttgggacagcaagcagaagctccttctgctgaacaattctctttagatgagagttatggggtggatgttaaccaaagaaaagacatgctttttgaagattttactcagcctctggccaatgctgaatctggcgaatctggggtgagcactaccattgctgtagaagagtcatacgggcttcctcttgagctgccctctgacctctctgtgcttacaactcgaagtcccactgtaagtaatcaaaatcatgggccacttatctcggaaacctctgaacgcaccatgttagctctggctacggaagagtcagaagctgggaaaagcaagaagaaaacaagaacggggtccactgtatccagcaagagcccacaggagggatgtgctgattcacaggttccagaaggacacatgactcctgaacacttcattcctccaagtgttgatggtgaccatattacaagccctggagtagcacctgtgggagagaccgggaaccaagatatgactagaactagtagcacgccagttcttcccagctcacccaccttgcctctccagaatcagaagttcatccctgctaccactgtcacctcaggtccggctccaattacaagttctgctgttcaagctgctgcctctcagttgaagcctggcccagagaaattgattgtgcttaaccaacatctgcagccactctatgtattgcaaaccgttcctaatggtgtcatgaatcctaatgcccctgtcttgacaggactcagtggtggcatctccacatctcagtccatttttcctgctggcagtaaaggtttagtgcctgtatctcatcatccacaaatccatgcattcacaggcaccactcagacaggtttccaaccagtcattcccagcaccacatctggcctgctcatgggagttacctcccatgatccccagattggtgtaacagaagcaggacataggcatgatcatgcccctaatgttgccatggtatctagtgcttcaactatcaccccagctccatccatgattccctctggtcatggcaaaaagcgccttatttcccgtcttcagagtcctaagagcaagaaacaggctcgcccaaaaacccagcccactcttgctccttctgatgttggacccaatatgaccctcattaatttgtcaccttcacagattgcagcaggcatccctgctcagacaggcctgatggaactggggactataactgccacacctcatcgaaaaattccaaacatcataaaacggccaaagcaaggagtgatgtacttggagcctactatcctcccacagcccatgcccatctcaaccacaactcagcctggcatactgggacatgattcctcaactcacctgctcccatgcactgtgtcggggctcaacacaagtcagtctgttttaaatgtagtgtcggtcccttccagtgcacctggaaactttttggggggcagctctgtatctctaagtgccccaggcctcattagctcaactgagatcacaggatctttaagtaacctccttatcaaagccaaccctcacaacctgagcctttcagagcaaccaatggttcttcatccaggaaccccaatgatgtctcatcttgcaaatcctgcccagacgtccattgccagtagcatttgtgtttttcccccaaaccaaagcataactgtgcctgtcaaccagcaagtggagaaggagggcactgtccatctccaacatgcggtcagtcgagtcctggcggataagacccttgacccaaatgtcagcccagctggtcaagtggctcttgcccctaatcctatctctcaagaacttaacaaaggtcatgttgttagcgtccttactcagagttcaagaacctctcccatctctcggccacaacatcagcaccaagcctcaaaattacctgctggagcaagctcagttgcgtttggaaaagggaaacataaagcaaaaagaccccgtccatgtccagataagagcagtggaaagaaacacaaaggacttcattcagatacaccaactgttgacacatctgcaatccaattatcatatattaaaggggaccaggaactgtcatcacctgagccaatggatacaggacagtctaatgaaaccggttcaaagaagagggattctaccactatgactaccaattcttctgctctgaaacgtaaaaccgtagatgctgttgatgagaaaccaagtactgcaggactgccaagtaaaggtgatggaacaggaaacaaagcattttcagtggatacacctgatcaaagggacagtgggagagattcttctctggaccacaagcccaagaaaggcctcatatttgaaatctgcagtgatgacggatttcagattcgctgtgagagtattgaggaggcctggaagtccctgacagataaagtgcaagaagctcggtccaatgctagactcaaggcactttctttcgatggagtaaatgggttgaagatgctgggtgtggttcatgatgctgtagtttttctgctggagcagctgtatggagccaggcattgccggaactataggtttcgcttccacaagcctgaggagacagactatcttcctgtaaaccctcatggatctgcccgtgctgaagtgtaccacaggaaatcagttttggatatgtttaatttcctggcatccaaacaccgtcagcctccagtatacaaccctcaggaggaagatgaggaggagatgcaacagaagtctgctcgacgggccaccagcacagacttgccactgcctgagaagttcaggcagttgaagaaagcatccagggacgctgtgggtgcctatagatcagccatacatggcagaggtttgttctgcaggaagaacattgagcccggagaaatggtgatcgagtattctggcaatgtaattcgttctgtcctcactgacaagcgggagaagtactatgatgacaagggcattggctgctacatgtttcgaatcgatgactacgaggtggtggatgctaccattcacggcaactcagcccgtttcattaaccactcatgcgagcccaactgctactctcatgtggtcaatgttgacggtcagaagcacattgtcatttttgccacacgcaggatctataaaggcgaggagctcacgtatgattacaagtttcccatcgaagaaccaggcaataagctgccttgcaactgcggggcaaagaagtgtcgcaagttcctcaattga

SEQ ID NO: 2 - protein, 4218 aa, Danio rerio
MAHSCRWRFPARPGGSSSSGTGRKAGRIRVNASLLISAGTNPNANGLGPGFDAALQVSAAIGSNLQKFRDVLGESSGSSSGEEEFGGFTTVSDNRRLHSPGRTSIGSITPDKKPRGRPPRTPAVQRVGTDAETAPLPVATSPTEKLKRQPGRPPGTREKKRGRPPASVSQRTWQHSGHALPEEGREVPQECSSSPVHSKEGVEENKEKRQTPLGSGHHQGSEAKLHKVSRESKVTKLKRLREVKLSPLKSKLKAIVRKTVTVPGKQRRKRGRPPSAERLKAEAAAAAAAAQAANASMAQETSTTAPRTAKKKAFRVRRSQDLGARTPHELRASHTADEHTHSDSQDSPTAADPLTPTKVGRPLGLRQSPRHIKPVRVVPPSKRTDATIAKQLLQRAKKGAQKKKLLEKDSVGTQGKAGLEAGKHRRRTQLTNIRQFIMPVVSTVSLRIIKTPKRFIEDEGSFSTPPPHMKIARLESALTAPAPQPATPSTPALVSTAPSTSGTTATPGSGSAVESLPPPPPPVSTGSTTAIAASLLNSSCNNSTSNGRFSSSAASCGSSAVSQHSSQLSSGEPSRSTSPSLDDSSCDSQASEGTQALSEEVDHSPASQGETEASLHHASHPPSPTSEPEPDHIVLEHSRRGRRGQSHRRGAVVARGRGNLIIGRKQAIISPATGVSQAGSQQASSTASSSSSPPPPPLLSPPQPPQTASSNAAEHHSHSPWMMSHSIAPFLPTSSILSSSHDKRRSILREPTFRWTSLSCAENKYFSSAKYAKEGLIRKPTFDNFRPPPLTAEDVGLMPPVTGGGGVTSGGFPAPGGAAGTGTRLFSPLHHHPHHNHHQHSSSRFETPLQKRTPLLRPPFFTPSPAHSRIFESVTLPSSSGSSPGSLSPLQVSPTSSKKKKGSRFPRGQPRSPSHSMITRSCQSGVPTGKSSEQSIISSSVPITVTGNSSPLPGVAVSPLAASALTQASFSGFPSGSIGLTSHGVSDGRRAAGGLSVSGNSASSSQLFPLFTPSPQASGGGTGKAGKERGISATRDTGTKEKDREMEKSREREKENKRDGRRDWDKRGKSLPSEASPSSISSFFGLEAIEESLTQKRTPGRKKSVTVDCAEASPSDSAAVQAVGSLSSKGRLTKKGRPPEKSIESEGVEREKDKEKLSALTQAGQMGKPPTTTSIDSILDRAEKRPVTDRRVVRLLKKAKAQLNKIEKRELQPGDQPKLPGQESDSSETSVRGPRIKHVCRRAAVALGRNRAVFPDDMPTLSALPWEEREKILSSMGNDDKSSVAGSEEAEPPTPPIKPVTRQKTVHEAPPRKGRRSRRCGQCPGCQVPNDCGVCTNCLDKPKFGGRNIKKQCCKVRKCQNLQWMPSKFLQKQAKGKKDRRRNKLSEKKELHHKSQCSEASPKSVPPPKDEPPRKKSETPPPAQGDDKQKQTQPSSPSSPASSPKDPLLSSPPDDHKHSLTSLSSACRKERKQQPSSSPTFLHAAPSSPPAQSQHSLQQPCQMPAKKEGLTKSQSHTEPKKKSQQQSQPSSATDTAPDAKLKKQTTRCVQPLKPKPKEKEKQLPKPDSSTLNSQSTPSTGGTAKQKAPYDGVHRIRVDFKEDYNIENVWEMGGLSILTSVPITPRVVCFLCASSGNVEFVFCQVCCEPFHLFCLGEAERPHDEQWENWCCRRCRFCHVCGRKYQKTKQLLECDKCRNSYHPECLGPNHPTRPTKKKRVWVCTKCVRCKSCGATKPGKAWDAQWSHDFSLCHDCAKRLTKGNLCPLCNKGYDDDDCDSKMMKCKKCDRWVHAKCESLTDDMCELMSSLPENVVYTCTNCTGSHPAEWRTVLEKEIQRSMRQVLTALFNSRTSTHLLRYRQAVMKPPELNPETEESLPSRRSPEGPDPPVLTEVSPPNDSPLDLESVEKKMDSGCYKSVLEFSDDIVKIIQTAFNSDGGQLESRKANSMLKSFFIRQMERIFPWYKVKESKFWETSKASSNSGLLPNVVLPPSLDHNYAQCQEREEMAKAGQSVHMKKIIPAPHPKAPGEPNSLMAPTPPPPPPMLIHDHSLEGSPVVPPPPGVGDNRQCALCLNYGDEKTNDCGRLLYIGHNEWAHVNCALWSAEVYEDVDGALKNVHMAVSRGKQLQCKNCHKPGATVSCCMTSCTNNYHFMYARQQQCAFLEDKKVYCQHHKDLVKGEVVPESSFEVTRRVLVDFEGIRLRRKFVNGLEPDNIHMVIGSMTIDCLGMLTELSDCERKLFPVGYQCSRVYWSTLDARKRCVYKCRILVCRPPLSETLNKNIAAQEENHTVIHSPPPVSVDTFLPGPIDSTKPSNVPSTPKPRVYFRNRHPSFPPCHRSPSTRPLPSPDGFNNTGHEIVTVGDPLLSSSLRSIGSRRHSTSSISAQQPRQKVSSPPQGGTVYSQTGNSSASFMSSTSKEPLTKDTDKGRVSSGETSFSREPNSINIGAQRRLSFGFTERVDGSKEATKKHSDGESLRSSQPASVSQVSPPLGTAVLTGHQRASGGIKNEKGKQATKDNDLPAGATFMSSHPLAMLPKDKANPNKEGNMTSMAALKDTVKTGSPQRIYNKSGSRKSHDYASGPAAVVAMKPLWSSGAKLGEEDIKRGFQASAGITGSHGTSSTKEKHSKVKMNVSRDVSKERKETPQNRNAVLNSNSKSSNVKTQGQVPPPHNISNKATALSSNTGSGTVEVNKFDQKEVEKPLKSKERFSFEKKHTSAMDAIQPKAGSERSIRPPQVHPKSSKEVPLVGKKHTERLSLMSQKMDPNRTKAVSISPNTQTYTSVTPSNQGPQRRSSRAMVFSPSASSESSESDSHIHPDDSEEHLMDHQCADDGEDNNLEDEGSVDKHHEEDSDGSAGSAKRRYPRRSARARSNMFFGLTPFYGVRSYGEEDIPFYRSGEISMKKRTGSSKRSAEGQVDGADDMSTSSSADSGEDEEGGIGSNKDTYYYNFTRTMINPSSGLPSIAGIDQCLGRGSQIHRFLRDQAKEHEDDSDEVSTATKNLELQQIGQLDGVDDGSESDISISTSSTTTATTSSTQKGSTKRKGRESRTEKSNVDSGKEAVNTTSNSRDSRKNQKDNCLPLGSAKTQGQDPLETQLSLTTDLLKSDSDNNNSDDCGNILPSDIMEFVLNTPSMQALGQQAEAPSAEQFSLDESYGVDVNQRKDMLFEDFTQPLANAESGESGVSTTIAVEESYGLPLELPSDLSVLTTRSPTVSNQNHGPLISETSERTMLALATEESEAGKSKKKTRTGSTVSSKSPQEGCADSQVPEGHMTPEHFIPPSVDGDHITSPGVAPVGETGNQDMTRTSSTPVLPSSPTLPLQNQKFIPATTVTSGPAPITSSAVQAAASQLKPGPEKLIVLNQHLQPLYVLQTVPNGVMNPNAPVLTGLSGGISTSQSIFPAGSKGLVPVSHHPQIHAFTGTTQTGFQPVIPSTTSGLLMGVTSHDPQIGVTEAGHRHDHAPNVAMVSSASTITPAPSMIPSGHGKKRLISRLQSPKSKKQARPKTQPTLAPSDVGPNMTLINLSPSQIAAGIPAQTGLMELGTITATPHRKIPNIIKRPKQGVMYLEPTILPQPMPISTTTQPGILGHDSSTHLLPCTVSGLNTSQSVLNVVSVPSSAPGNFLGGSSVSLSAPGLISSTEITGSLSNLLIKANPHNLSLSEQPMVLHPGTPMMSHLANPAQTSIASSICVFPPNQSITVPVNQQVEKEGTVHLQHAVSRVLADKTLDPNVSPAGQVALAPNPISQELNKGHVVSVLTQSSRTSPISRPQHQHQASKLPAGASSVAFGKGKHKAKRPRPCPDKSSGKKHKGLHSDTPTVDTSAIQLSYIKGDQELSSPEPMDTGQSNETGSKKRDSTTMTTNSSALKRKTVDAVDEKPSTAGLPSKGDGTGNKAFSVDTPDQRDSGRDSSLDHKPKKGLIFEICSDDGFQIRCESIEEAWKSLTDKVQEARSNARLKALSFDGVNGLKMLGVVHDAVVFLLEQLYGARHCRNYRFRFHKPEETDYLPVNPHGSARAEVYHRKSVLDMFNFLASKHRQPPVYNPQEEDEEEMQQKSARRATSTDLPLPEKFRQLKKASRDAVGAYRSAIHGRGLFCRKNIEPGEMVIEYSGNVIRSVLTDKREKYYDDKGIGCYMFRIDDYEVVDATIHGNSARFINHSCEPNCYSHVVNVDGQKHIVIFATRRIYKGEELTYDYKFPIEEPGNKLPCNCGAKKCRKFLN

SEQ ID NO: 3 - DNA, 561 bp, Danio rerio
tgggatcctgtcggggtcctcggtaccaccgccccgaaacatggataatccattttgaccgctagacgcgagctgccgtgtgctgagatcgctcgttcggggctacacccacactgagctcctgatcctagggcaggcaggcagagtgtaaaatggcgcacagctgtcggtggcggttccctgctcggcccggagggagcagcagctcgggcaccgggaggaaagcgggccgaattcgagtcaatgcttccctccttatcagcgcgggaacaaatccgaacgcgaacgggctcgggcccggtttcgacgctgcgttgcaagtgtccgctgccatcggcagcaacctgcagaaatttcgggatgttttgggggagtcgagcggctccagtagcggggaggaggaatttggaggctttaccacagttagtgacaacagaagactacatagccctggaagaacgtcaataggttctatcacaccagacaagaagcccagaggacgtcctcctaggactcctgctgtgcagagggttggcactgatgctgaaacagcccctttgc

SEQ ID NO: 4 - DNA, 152 bp, Danio rerio
tgggatcctgtcggggtcctcggtaccaccgccccgaaacatggataatccattttgaccgctagacgcgagctgccgtgtgctgagatcgctcgttcggggctacacccacactgagctcctgatcctagggcaggcaggcagagtgtaaa

SEQ ID NO: 5 - protein, 129 aa, Homo sapien
RGLFCKRNIDAGEMVIEYAGNVIRSIQTDKREKYYDSKGIGCYMFRIDDSEVVDATMHGNRARFINHSCEPNCYSRVINIDGQKHIVIFAMRKIYRGEELTYDYKFPIEDASNKLPCNCGAKKCRKFLN

SEQ ID NO: 6 - protein, 129 aa, Mus
RGLFCKRNIDAGEMVIEYAGNVIRSIQTDKREKYYDSKGIGCYMFRIDDSEVVDATMHGNAARFINHSCEPNCYSRVINIDGQKHIVIFAMRKIYRGEELTYDYKFPIEDASNKLPCNCGAKKCRKFLN

SEQ ID NO: 7 - protein, 129 aa, Fugu
RGLFCKKTIEAGEMVIEYSGNVIRSVLTDKREKYYDGKGIGCYMFRIDDYEVVDATVHGNAARFINHSCEPNCYSRVITVDGKKHIVIFASRRIYRGEELTYDYKFPIEDASSKLPCNCNSKKCRKFLN

SEQ ID NO: 8 - protein, 127 aa, Drosophila
RGLYCTKDIEAGEMVIEYAGELIRSTLTDKRERYYDSRGIGCYMFKIDDNLVVDATMRGNAARFINHCCEPNCYSKVVDILGHKHIIIFALRRIVQGEELTYDYKFPFEDEKIPCSCGSKRCRKYLN

SEQ ID NO: 9 - protein, 8 aa, Artificial Sequence
  Synthetic Sequence
AGEMVIEY

SEQ ID NO: 10 - DNA, 24 bp, Artificial Sequence
  Synthetic Sequence
gcaggtgagatggtgattgagtat

SEQ ID NO: 11 - DNA, 24 bp, Artificial Sequence
  Synthetic Sequence
gcaggagagatggtgattgaatac

SEQ ID NO: 12 - DNA, 24 bp, Artificial Sequence
  Synthetic Sequence
gctggtgaaatggtcattgaatat

SEQ ID NO: 13 - DNA, 24 bp, Artificial Sequence
  Synthetic Sequence
gcgggtgaaatggttatcgaatat

SEQ ID NO: 14 - DNA, 24 bp, Artificial Sequence
  Primer
gcdggwgaratggtbatygartay

SEQ ID NO: 15 - DNA, 24 bp, Artificial Sequence
  Primer
gcdggtgaratggtbattgaatat

SEQ ID NO: 16 - DNA, 20 bp, Artificial Sequence
  Primer
gagagcaggaaagccaacag

SEQ ID NO: 17 - DNA, 27 bp, Artificial Sequence
  Primer
tggttcaagtccattaacaaattttct

SEQ ID NO: 18 - DNA, 21 bp, Artificial Sequence
  Primer
tttggctgacagaagcaggag

SEQ ID NO: 19 - DNA, 20 bp, Artificial Sequence
  Primer
gcaaaggggctgtttcagta

SEQ ID NO: 20 - DNA, 25 bp, Artificial Sequence
  Primer
aatttcgggatgttttgggggagtc

SEQ ID NO: 21 - DNA, 25 bp, Artificial Sequence
  Primer
agcttattgcctggttcttcgatgg

SEQ ID NO: 22 - protein, 1114 aa, Homo Sapien
PVTRNKAPQEPPVKKGRRSRRCGQCPGCQVPEDCGVCTNCLDKPKFGGRNIKKQCCKMRKCQNLQWMPSKAYLQKQAKAVKKKEKKSKTSEKKDSKESSVVKNVVDSSQKPTPSAREDPAGVHRIRVDFKEDCEAENVWEMGGLGILTSVPITPRVVCFLCASSGHVEFVYCQVCCEPFHKFCLEENERPLEDQLENWCCRRCKFCHVCGRQHQATKQLLECNKCRNSYHPECLGPNYPTKPTKKKKVWICTKCVRCKSCGSTTPGKGWDAQWSHDFSLCHDCAKLFAKGNFCPLCDKCYDDDDYESKMMQCGKCDRWVHSKCENLSDEMYEILSNLPESVAYTCVNCTERHPAEWRLALEKELQISLKQVLTALLNSRTTSHLLRYRQAAKPPDLNPETEESIPSRSSPEGPDPPVLTEVSKQDDQQPLDLEGVKRKMDQGNYTSVLEFSDDIVKIIQAAINSDGGQPEIKKANSMVKSFFIRQMERVFPWFSVKKSRFWEPNKVSSNSGMLPNAVLPPSLDHNYAQWQEREENSHTELCLTYGDDSANDAGRLLYIGQNEWTHVNCALWSAEVFEDDDGSLKNVHMAVIRGKQLRCEFCQKPGATVGCCLTSCTSNYHFMCSRAKNCVFLDDKKVYCQRHRDLIKGEVVPENGFEVFRRVFVDFEGISLRRKFLNGLEPENIHMMIGSMTIDCLGILNDLSDCEDKLFPIGYQCSRVYWSTTDARKRCVYTCKIVECRPPVVEPDINSTVEHDENRTIAHSPTSFTESSSKESQNTASTGKKRGKRSAEGQVDGADDLSTSDEDDLYYYNFTRTVISSGGEERLASHNLFREEEQCDLPKISQLDGVDDGTWLQQEQKRKESITEKKPKKGLVFEISSDDGFQICAESIEDAWKSLTDKVQEARSNARLKQLSFAGVNGLRMLGILHDAVVFLIEQLSGAKHCRNYKFRFHKPEEANEPPLNPHGSARAEVEAVGVYRSPIHGRGLFCKRNIDAGEMVIEYAGNVIRSIQTDKREKYYDSKGIGCYMFRIDDSEVVDATMHGNRARFINHSCEPNCYSRVINIDGQKHIVIFAMRKIYRGEELTYDYKFPIEDASNKLPCNCGAKKCRKFLN

SEQ ID NO: 23 - protein, 1152 aa, Danio rerio
PVTRQKTVHEAPPRKGRRSRRCGQCPGCQVPNDCGVCTNCLDKPKFGGRNIKKQCCKVRKCQNLQWMPSKFLQKQAKGKKDRRRNKLSEKKELHHKSQCSEASPKSVPPPKDEPPGVHRIRVDFKEDYNIENVWEMGGLSILTSVPITPRVVCFLCASSGNVEFVFCQVCCEPFHLFCLGEAERPHDEQWENWCCRRCRFCHVCGRKYQKTKQLLECDKCRNSYHPECLGPNHPTRPTKKKRVWVCTKCVRCKSCGATKPGKAWDAQWSHDFSLCHDCAKRLTKGNLCPLCNKGYDDDDCDSKMMKCKKCDRWVHAKCESLTDDMCELMSSLPENVVYTCTNCTGSHPAEWRTVLEKEIQRSMRQVLTALFNSRTSTHLLRYRQAVMKPPELNPETEESLPSRRSPEGPDPPVLTEVSPPNDSPLDLESVEKKMDSGCYKSVLEFSDDIVKIIQTAFNSDGGQLESRKANSMLKSFFIRQMERIFPWYKVKESKFWETSKASSNSGLLPNVVLPPSLDHNYAQCQEREEMAKAGLCLNYGDEKTNDCGRLLYIGHNEWAHVNCALWSAEVYEDVDGALKNVHMAVSRGKQLQCKNCHKPGATVSCCMTSCTNNYHFMYARQQQCAFLEDKKVYCQHHKDLVKGEVVPESSFEVTRRVLVDFEGIRLRRKFVNGLEPDNIHMVIGSMTIDCLGMLTELSDCERKLFPVGYQCSRVYWSTLDARKRCVYKCRILVCRPPLSETLNKNIAAQEENHTVIHSPPPVSVDTFLPGPGEISMKKRTGSSKRSAEGQVDGADDMSTSSSADSGEDEEGGIGSNKDTYYYNFTRTMINPSSGLPSIAGIDQCLGRGSQIHRFLRDQAKEHEDDSDEVSTATKNLELQQIGQLDGVDDGSTPDQRDSGRDSSLDHKPKKGLIFEICSDDGFQIRCESIEEAWKSLTDKVQEARSNARLKALSFDGVNGLKMLGVVHDAVVFLLEQLYGARHCRNYRFRFHKPEETDYLPVNPHGSARAEVDAVGAYRSAIHGRGLFCRKNIEPGEMVIEYSGNVIRSVLTDKREKYYDDKGIGCYMFRIDDYEVVDATIHGNSARFINHSCEPNCYSHVVNVDGQKHIVIFATRRIYKGEELTYDYKFPIEEPGNKLPCNCGAKKCRKFLN

SEQ ID NO: 24 - protein, 5 aa, Artificial Sequence
  Synthetic Sequence
DGVDD

SEQ ID NO: 25 - protein, 5 aa, Artificial Sequence
  Synthetic Sequence
DGADD

SEQ ID NO: 26 - DNA, 20 bp, Artificial Sequence
  Primer
cttcatccaatgcagcagaa

SEQ ID NO: 27 - DNA, 20 bp, Artificial Sequence
  Primer
gtgggcggaaatatcagaaa

SEQ ID NO: 28 - DNA, 20 bp, Artificial Sequence
  Primer
caggttgatggagcagatga

SEQ ID NO: 29 - DNA, 20 bp, Artificial Sequence
  Primer
aagctcggtccaatgctaga

SEQ ID NO: 30 - DNA, 20 bp, Artificial Sequence
  Primer
cgtgatgaggaatgttggtg

SEQ ID NO: 31 - DNA, 20 bp, Artificial Sequence
  Primer
tctctgggttgagctcaggt

SEQ ID NO: 32 - DNA, 20 bp, Artificial Sequence
  Primer
tttactgcctccttccctga

SEQ ID NO: 33 - DNA, 20 bp, Artificial Sequence
  Primer
cgcttgtcagtgaggacaga

SEQ ID NO: 34 - DNA, 20 bp, Artificial Sequence
  Primer
caaccctcaggaggaagatg

SEQ ID NO: 35 - DNA, 20 bp, Artificial Sequence
  Primer
cctgcagaacaaacctctgc

SEQ ID NO: 36 - DNA, 19 bp, Artificial Sequence
  Primer
cgagcaggagatgggaacc

SEQ ID NO: 37 - DNA, 19 bp, Artificial Sequence
  Primer
caacggaaacgctcattgc

SEQ ID NO: 38 - DNA, 50 bp, Artificial Sequence
  Synthetic Sequence
catagccctggaagaacgtcaataggtaaacaaattctctaaattattgt

SEQ ID NO: 39 - DNA, 25 bp, Artificial Sequence
  Synthetic Sequence
tagagaatttgtttacctattgacg

SEQ ID NO: 40 - DNA, 20 bp, Artificial Sequence
  Primer
ccgaattcgagtcaatgctt

SEQ ID NO: 41 - DNA, 21 bp, Artificial Sequence
  Primer
tttggctgacagaagcaggag

(19) United States
(12) Patent Application Publication
(10) Pub. No.: US 2009/0055940 A1
(43) Pub. Date: Feb. 26, 2009

(54) ZEBRAFISH MODEL OF MLL LEUKEMOGENESIS

(76) Inventors: CAROLYN A. FELIX, ARDMORE, PA (US); RITA BALICE-GORDON, GLEN MILLS, PA (US); GIUSEPPE GERMANO, PADUA (IT); YUAN-QUAN SONG, PHILADELPHIA, PA (US); BLAINE W. ROBINSON, EAST LANSDOWNE, PA (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/764,568

(22) Filed: Jun. 18, 2007

Related U.S. Application Data
(60) Provisional application No. 60/891,656, filed on Feb. 26, 2007.
(60) Provisional application No. 60/814,373, filed on Jun. 16, 2006.

(51) Int. Cl. A01K 67/027; C12N 15/11; C12N 15/00; C07K 14/00

(52) U.S. Cl. 800/3; 536/23.5; 435/320.1; 530/350; 800/10

(57) ABSTRACT

The zebrafish mll gene and methods of use thereof are provided.

This application claims priority under 35 U.S.C. §119(e) to U.S. Provisional Patent Application No. 60/891,656, filed on Feb. 26, 2007, and U.S. Provisional Patent Application No. 60/814,373, filed on Jun. 16, 2006. The foregoing applications are incorporated by reference herein.

### FIELD OF THE INVENTION

The present invention relates to a zebrafish model of MLL (Mixed Lineage Leukemia; Myeloid Lymphoid Leukemia) in hematopoiesis and leukemia.

### BACKGROUND OF THE INVENTION

Balanced chromosomal translocations of the MLL (Mixed Lineage Leukemia; Myeloid Lymphoid Leukemia) gene at chromosome band 11q23 are the primary genetic aberrations underlying most cases of acute leukemia in infants (Gilliland et al. (2004) Hematology (Am Soc Hematol Educ Program), 80-97). MLL translocations also are the most common of the balanced translocations in treatment-related leukemias after chemotherapeutic topoisomerase II poisons (Rowley et al. (2002) Genes Chromosomes Cancer, 33:331-45), and they comprise 5-10% of acquired chromosomal rearrangements in childhood and adult ALL and AML (Pui et al. (2003) Leukemia, 17:700-6; Bacher et al. (2005) Haematologica, 90:1502-10; Mancini et al. (2005) Blood, 105:3434-41). MLL translocations dictate distinctive biological properties and clinical behaviors Gilliland et al. (2004) Hematology (Am Soc Hematol Educ Program), 80-97).

Biologically, MLL translocations determine differentiation, lineage and immunophenotype of leukemia blast cell populations. For example, the blast populations in the ALL cases exhibit an early CD10-CD24-pro-B cell immunophenotype and uniquely co-express the myeloid associated antigen CD15 (Borkhardt et al. (2002) Leukemia, 16:1685-90). MLL translocations are strongly associated with myelomonocytic and monoblastic AML in infants and young children (Pui et al. (1995) Leukemia, 9:762-9) and in the treatment-related cases (Ratain et al. (1987) Blood, 70:1412-7; Pui et al. (1988) J. Clin. Oncol., 6:1008-13); however, leukemias with MLL translocations also can present as other AML morphologic subtypes or myelodysplastic syndrome (Smith et al. (1994) Med. Pediatr. Oncol., 23:86-98; Felix et al. (1995) Blood, 85:3250-6; Winick et al. (1993) J. Clin. Oncol., 11:209-17). This morphologic and phenotypic heterogeneity is influenced by the partner genes involved (Hunger et al. (1992) J. Clin. Oncol., 10:156-63; Sobulo et al. (1997) PNAS, 94:8732-7; Rowley et al. (1997) Blood, 90:535-41). Zebrafish may provide an evolutionary framework for a deeper understanding of the cell of origin and mixed lineage nature of leukemias with MLL translocations because a population of B cells has been observed in rainbow trout, a different teleost fish, with phagocytic properties classically ascribed to the monocyte/macrophage lineage (Li et al. (2006) Nat. Immunol., 7:1116-24).

In all of these patient populations, MLL translocations are poor prognostic factors with significant adverse effects on response to treatment. Chemotherapy resistance and toxic deaths contribute to a grave prognosis in infant leukemias with MLL translocations (Reaman G H (2003) Biology and treatment of acute leukemia in infants in treatment of acute leukemias. In: Pui C-H, editor. New Directions in Clinical Research: Humana Press, p. 73-83). Similarly, secondary leukemias with MLL translocations have a poor prognosis and limited treatment options. For an ultra high-risk population within infant ALL, the constellation of poor prognostic features including age <3 months at diagnosis, WBC count >100,000/μL, early pro-B CD10-immunophenotype and t(4;11) translocation, is associated with event free survival of ˜5% (Reaman et al. (1999) J. Clin. Oncol., 17:445-55; Reaman et al. (1985) J. Clin. Oncol., 3:1513-21). Infant leukemias with MLL translocations often are resistant to common chemotherapeutic agents (Pieters et al. (1998) Leukemia, 12:1344-8; Pui et al. (2002) Lancet, 359:1909-15). Infants also are more vulnerable to toxicities, and more intensive treatment for infant ALL has increased treatment complications without improving outcome (Hilden et al. (2006) Blood, 108:441-51). Event free survival rates in infant AML are ˜50% using current intensive treatments (Woods et al. (2001) Blood, 97:56-62). MLL translocations strongly predict poor clinical outcome and portend a grave prognosis in secondary leukemia also (Rowley et al. (2002) Genes Chromosomes Cancer, 33:331-45). Prognosis in the secondary cases is affected further by the limited feasibility of administering additional intensive anti-leukemia therapy after primary cancer treatment (Barnard et al. (2002) Blood, 100:427-34).

The MLL gene encodes a large, complex oncoprotein that regulates transcription (Rasio et al. (1996) Cancer Res., 56:1766-9; Djabali et al. (1992) Nature Genet., 2:113-8; Gu et al. (1992) Cell, 71:701-8; Tkachuk et al. (1992) Cell, 71:691-700; Ma et al. (1993) PNAS, 90:6350-4; Domer et al. (1993) PNAS, 90:7884-8). MLL was also originally named HRX and Htrx1 because its speckled nuclear localization (SNL) domains, plant homeodomains (PHDs) and SET domain have regional amino acid similarity to Drosophila trithorax (trx) (Djabali et al. (1992) Nature Genet., 2:113-8; Tkachuk et al. (1992) Cell, 71:691-700; Ayton et al. (2001) Oncogene, 20:5695-707). Drosophila trx group (trxG) and Polycomb-group (PcG) proteins, respectively, maintain expression or repression of homeotic gene complexes during embryonic development (Yu et al. (1998) PNAS, 95:10632-6; Mahmoudi et al. (2001) Oncogene, 20:3055-66). The trxG proteins are not required for transcription initiation but maintain transcription through later stages of development (Hanson et al. (1999) PNAS, 96:14372-7). MLL and BMI-1, mammalian homologues of trxG and PcG proteins, are antagonistic regulators of HOX gene expression (Hanson et al. (1999) PNAS, 96:14372-7). MLL maintains HOX gene expression during skeletal, craniofacial and neural development and hematopoiesis (Yu et al. (1998) PNAS, 95:10632-6; Yu et al. (1995) Nature, 378:505-8; Hess et al. (1997) Blood, 90:1799-806).

Constructs comprising MLL AT hook motifs have been shown to promote p21 and p27 upregulation, cell cycle arrest and monocyte differentiation (Caslini et al. (2000) PNAS, 97:2797-802). The amino terminal SNL motifs direct MLL subnuclear localization (Ayton et al. (2001) Oncogene, 20:5695-707). The cysteine-rich CXXC region is similar to the CXXC region in DNA methyltransferase 1 that recognizes CpG di-nucleotides (Lee et al. (2001) J. Biol. Chem., 276:44669-76). The MT domain is part of a transcriptional repression region (Ayton et al. (2001) Oncogene, 20:5695-707; Caslini et al. (2000) PNAS, 97:2797-802; Xia et al. (2003) PNAS, 100:8342-7; Yokoyama et al. (2002) Blood, 100:3710-8). The PHD mediates MLL homodimerization and protein interactions including binding to a nuclear cyclophilin, which modulates target gene expression (Fair et al. (2001) Mol. Cell. Biol., 21:3589-97). The SET domain interacts with the SWI/SNF chromatin remodeling complex, which activates transcription (Rozenblatt-Rosen et al. (1998) PNAS, 95:4152-7). Consistent with its role in epigenetic gene regulation, the SET domain has specific histone H3 lysine-4-specific methyltransferase activity that regulates HOX promoters (Milne et al. (2002) Mol. Cell., 10:1107-17).

Taspase 1 cleaves MLL into an amino terminal fragment with transcriptional repression properties and a carboxyl terminal fragment with transcriptional activation properties, which associate with one another and other chromatin regulatory proteins in a large protein complex (Yokoyama et al. (2002) Blood, 100:3710-8; Hsieh et al. (2003) Cell, 115:293-303). MLL proteolytic cleavage by taspase1 and association of its N and C terminal fragments is critical for proper nuclear sublocalization and HOX gene regulation (Hsieh et al. (2003) Cell, 115:293-303). In addition, MLL proteolytic cleavage is essential for cell cycle progression (Takeda et al. (2006) Genes Dev., 20:2397-409), some implications of which will be elaborated in the zebrafish model.

MLL translocations disrupt an 8.3 kb breakpoint cluster region between exons 5-11 and involve >50 partner genes that encode diverse partner proteins (Rowley, J D (1998) Annu. Rev. Genet., 32:495-519; Felix, Calif. (2000) Hematology 2000: Education Program of the American Society of Hematology 2000:294-8; Ayton et al. (2001) MLL in Normal and Malignant Hematopoiesis. In: Ravid K, Licht J D, editors. Transcription Factors: Normal and Malignant Development of Blood Cells. New York: Wiley-Liss, Inc.; Huret, J L. (1998) Leukemia, 12:811-22). Many MLL partner proteins have structural motifs of nuclear transcription factors (Gu et al. (1992) Cell, 71:701-8; Tkachuk et al. (1992) Cell, 71:691-700; Morrissey et al. (1993) Blood 81:1124-31; Taki et al. (1996) Oncogene 13:2121-30; Taki et al. (1999) PNAS, 96:14535-40; Hillion et al. (1997) Blood, 9:3714-9; Chaplin et al. (1995) Blood 86:2073-6; Schichman et al. (1994) PNAS, 91:6236-9; Prasad et al. (1994) PNAS, 91:8107-11; Nakamura et al. (1993) PNAS, 90:4631-5; Borkhardt et al. (1997) Oncogene 14:195-202), transcriptional regulatory proteins (Sobulo et al. (1997) PNAS, 94:8732-7; Taki et al. (1997) Blood, 89:3945-50; Thirman et al. (1994) PNAS, 91:12110-4; Ida et al. (1997) Blood, 90:4699-704) or other nuclear proteins (Ono et al. (2002) Cancer Res., 62:4075-80; Lorsbach et al. (2003) Leukemia 17:637-41; Hayette et al. (2000) Oncogene 19:4446-50). Others are cytoplasmic proteins (Bernard et al. (1994) Oncogene 9:1039-45; Tse et al. (1995) Blood 85:650-6; Sano et al. (2000) Blood 95:1066-8; Pegram et al. (2000) Blood 96:4360-2; Daheron et al. (2001) Genes Chromosomes & Cancer 31:382-9; Borkhardt et al. (2000) PNAS, 97:9168-73; Raffini et al. (2002) PNAS, 99:4568-73; Fuchs et al. (2001) PNAS, 98:8756-61; Taki et al. (1998) Blood 92:1125-30; Fu et al. (2003) Genes, Chromosomes & Cancer 37:214-19; Chinwalla et al. (2003) Oncogene 22:1400-10; Megonigal et al. (2000) PNAS, 97:2814-9; Strehl et al. (2003) Oncogene 22:157-60; So et al. (1997) PNAS 99:2563-8; Megonigal et al. (1998) PNAS, 95:6413-8; Osaka et al. (1999) PNAS, 96:6428-33; Taki et al. (1999) Cancer Res 59:4261-5; Borkhardt et al. (2001) Genes Chromosomes & Cancer 32:82-8; Ono et al. (2002) Cancer Res., 62:333-7; Slater et al. (2002) Oncogene 21:4706-14), cell membrane proteins or proteins in different cellular locations (Eguchi et al. (2001) Genes Chromosomes Cancer 32:212-21; Wechsler et al. (2003) Genes, Chromosomes & Cancer 36:26-36; Kourlas et al. (2000) PNAS, 97:2145-50; Prasad et al. (1993) Cancer Res., 53:5624-8; LoNigro et al. (2002) Blood 100(Suppl 1):531a). MLL also undergoes self-fusions and MLL itself is a partner protein (Schichman et al. (1994) PNAS, 91:6236-9; Caligiuri et al. (1996) Cancer Res., 56:1418-25; Megonigal et al. (1997) PNAS, 94:11583-8). While some MLL partner genes are members of the same gene families (Ayton et al. (2001) MLL in Normal and Malignant Hematopoiesis. In: Ravid K, Licht J D, editors. Transcription Factors: Normal and Malignant Development of Blood Cells. New York: Wiley-Liss, Inc.; 2001; Huret, J L (2001) 11q23 rearrangements in leukaemia. In: Atlas Genet Cytogenet Oncol Haematol; Taki et al. (1999) PNAS, 96:14535-40; Megonigal et al. (1998) PNAS, 95:6413-8; Osaka et al. (1999) PNAS, 96:6428-33; Taki et al. (1999) Cancer Res., 59:4261-5; Borkhardt et al. (2001) Genes Chromosomes & Cancer 32:82-8; Ono et al. (2002) Cancer Res 62:333-7; Slater et al. (2002) Oncogene 21:4706-14; Nilson et al. (1997) Br. J. Haematol., 98:157-69; Tatsumi et al. (2001) Genes Chromosomes & Cancer 30:230-5) or encode proteins with otherwise similar functions (Hillion et al. (1997) Blood 9:3714-9; Borkhardt et al. (1997) Oncogene 14:195-202; So et al. (2002) Mol. Cell. Biol., 22:6542-52; So et al. (2003) Blood 101:633-9), there is no unifying functional relationship between the many partner genes. The most common MLL partner genes are AF4, ENL and AF9 (Secker-Walker, L M (1998) Leukemia 12:776-8). In ALL, the partner genes are limited and AF4 is the most common, whereas in AML the partner genes are much more diverse. The partner genes in de novo and treatment-related leukemias are at least partially overlapping. Of interest also is that some of the MLL partner proteins such as AF4 and AF9 interact with one another (Erfurth et al. (2004) Leukemia 18:92-102).

Fusion proteins from the der(11) chromosome, which retain the AT-hook, SNL and MT domains of MLL but replace the MLL PHD, transactivation, and SET domains with the carboxyl partner protein, transform hematopoietic progenitors and cause leukemia in mice (Ayton et al. (2001) MLL in Normal and Malignant Hematopoiesis. In: Ravid K, Licht J D, editors. Transcription Factors: Normal and Malignant Development of Blood Cells. New York: Wiley-Liss, Inc.; Corral et al. (1996) Cell 85:853-61; Lavau et al. (1997) Embo J 16:4226-37; Lavau et al. (2000) PNAS, 97:10984-9; Lavau et al. (2000) Embo J., 19:4655-64; So et al. (2003) Cancer Cell 3:161-71; Liedman et al. (2001) Curr. Opin. Hematol., 8:218-23). The der(11) fusion proteins lack the taspase1 site and cannot interact with the MLL C terminus (Yokoyama et al. (2002) Blood 100:3710-8; Hsieh et al. (2003) Cell 115:293-303.). Murine models of MLL fusion oncoproteins have suggested that the function of nuclear partner proteins involves transcriptional activation (Ayton et al. (2001) Oncogene 20:5695-70730; So et al. (2003) Blood 101:633-9; Zeisig et al. (2003) Leukemia 17:359-65), whereas cytoplasmic partner proteins result in forced MLL dimerization or oligomerization (So et al. (2003) Cancer Cell 4:99-110). Murine models have also demonstrated that MLL fusion proteins constitutively activate Hoxa9 and that Hoxa9 activation is essential for leukemia with some MLL fusion proteins (e.g. MLL-ENL) (Ayton et al. (2003) Genes Dev., 17:2298-307). However, altered Hox expression influences phenotype, latency and penetrance, but is not essential with other MLL fusion proteins (e.g. MLL-AF9, MLL-GAS-7) (Kumar et al. (2004) Blood 103:1823-8; So et al. (2004) Blood 103:3192-9).

In infant leukemias the MLL translocation is an acquired, in utero alteration and there is a short latency to the diagnosis of leukemia during the first year of life (Megonigal et al. (1998) PNAS 95:6413-8; Gale et al. (1997) PNAS 94:13950-4; Ford et al. (1993) Nature 363:358-60). In treatment-related leukemias with MLL translocations the typical latency is about two years after the chemotherapy exposure (Smith et al. (1994) Med. Pediatr. Oncol., 23:86-98). Latency to leukemia in patients and in mice has suggested that secondary alterations may be important in addition to the translocations for leukemia to occur (Ayton et al. (2001) Oncogene 20:5695-707; Ayton et al. (2001) MLL in Normal and Malignant Hematopoiesis. In: Ravid K, Licht J D, editors. Transcription Factors: Normal and Malignant Development of Blood Cells. New York: Wiley-Liss, Inc.).

While some functions of MLL and MLL fusion proteins have been clarified, the many partner genes have made the role of the fusion proteins complex to resolve. The significance of disruption of partner proteins with key roles in cellular functions, and of fusion proteins predicted by the der(other) chromosomes have yet to be fully resolved (Raffini et al. (2002) Proc. Natl. Acad. Sci., 99:4568-73). The zebrafish model of the instant invention has advantages to uncover novel cellular programs controlled by MLL and deregulated by the fusion proteins.

Zebrafish (Danio rerio) models offer many advantages for developmental and genetic studies including high fecundity, short generation time and small size at maturation (Hsu et al. (2001) Curr. Opin. Hematol., 8:245-51). The rapid, easily visualized, external development of transparent embryos enables real-time functional observations of hematopoietic development unlike any other models, and blood circulation in zebrafish becomes visible under the microscope by 24 hours postfertilization (hpf) (de Jong et al. (2005) Annu. Rev. Genet., 39:481-501). Large segments of zebrafish chromosomes are syntenic with human and mouse genomes (Barbazuk et al. (2000) Genome Res., 10:1351-8). Moreover, many mammalian genes have zebrafish orthologs and they have evolved from the same ancestral genes sharing common functions (Barbazuk et al. (2000) Genome Res., 10:1351-8). Many zebrafish orthologs of blood-specific genes have also been isolated (e.g. cmyb, gata1, gata2, globin, ikaros, lmo2, pu.1, rag1, rag2, runx1, cbfb, and scl) (Hogan et al. (2006) Dev. Genes Evol.; Juarez et al. (2005) J. Biol. Chem., 280:41636-44; Galloway et al. (2005) Dev. Cell 8:109-16; Rhodes et al. (2005) Dev. Cell 8:97-108; Gering et al. (2003) Development 130:6187-99; Nishikawa et al. (2003) Mol. Cell. Biol., 23:8295-305; Blake et al. (2000) Blood 96:4178-84; Willett et al. (2001) Dev. Dyn., 222:694-8; Burns et al. (2002) Exp. Hematol., 30:1381-9). Gene expression profiling of kidney marrow cells, the site of definitive hematopoiesis in teleosts, has demonstrated that the genetic programs controlling hematopoiesis, angiogenesis and hematopoietic cell function are highly conserved from zebrafish to humans (Song et al. (2004) PNAS 101:16240-5).

Histochemical staining of hematopoietic cells and molecular analyses using whole mount in situ hybridization have aided greatly in characterizing the development of blood lineages in zebrafish. Zebrafish hematopoiesis and blood cell morphology closely parallel those of mammals (Galloway et al. (2003) Curr. Top. Dev. Biol., 53:139-58). In mammals, primitive hematopoiesis is largely erythropoietic and extra-embryonic in blood islands of the yolk sac. Later in embryogenesis, mammalian hematopoiesis moves to the aorta-gonad-mesonephros (AGM) and the fetal liver (Medvinsky et al. (1996) Cell 86:897-906), whereas definitive hematopoiesis occurs in the bone marrow where all blood cell lineages are produced (Johnson et al. (1975) Nature 258:726-8). Zebrafish lack extra-embryonic yolk sac blood islands and primitive hematopoiesis occurs within the intermediate cell mass (ICM) between notochord and endoderm, anteriorly over the yolk cell in the anterior lateral mesoderm (ALM) and posteriorly in a small ventral cluster of cells called posterior lateral mesoderm (PLM) (Thompson et al. (1998) Dev. Biol., 197:248-69; Detrich et al. (1995) PNAS 92:10713-7). By 10-12 hours post fertilization (hpf) the PLM expresses scl, gata2 and lmo2, indicating the formation of hematopoietic stem cells (HSCs) (Davidson et al. (2003) Nature 425:300-6; Davidson et al. (2004) Oncogene 23:7233-46). At 12-20 hpf initiation of erythropoiesis is marked by gata1 expression in a subset of scl+ cells in the PLM, whereas myelopoiesis and granulopoiesis, marked by myeloid-specific gene expression (e.g. pu.1, l-plastin) begins in the ALM (Bennett et al. (2001) Blood 98:643-51). Thus, the PLM and ALM give rise to erythroid and myeloid cells, respectively. By 24 hpf, proerythroblasts from the ICM expressing gata1 and embryonic globins begin to enter circulation (de Jong et al. (2005) Annu. Rev. Genet., 39:481-501).

By 31 hpf, expression of zebrafish c-myb and runx1 orthologs on HSCs herald definitive hematopoiesis in the kidney, and definitive HSCs subsequently colonize the thymus and pancreas (Davidson et al. (2004) Oncogene 23:7233-46). By >96 hpf myelopoiesis occurs in the kidney and the spleen as indicted by MPO+, PAS−, Acid Phosphatase+ cells and mpo and pu.1 gene expression (Crowhurst et al. (2002) Int. J. Dev. Biol., 46:483-92.). At 5 dpf, erythrocytes and granulocytes are produced in the kidney and by 13 dpf onward the kidney marrow is the primary hematopoietic organ (Willett et al. (1999) Dev. Dyn., 214:323-36; Weinstein et al. (1996) Development 123:303-9). However, zebrafish have only two granulocyte lineages, one resembling mammalian neutrophils and the second, produced in the spleen and kidney, with features of both mammalian eosinophils and basophils (Bennett et al. (2001) Blood 98:643-51; Herbomel et al. (1999) Development 126:3735-45; Lieschke et al. (2001) Blood 98:3087-96). Monocyte/macrophages expressing c-myb and l-plastin but not the neutrophil marker mpo have been identified in zebrafish embryos by 12-20 hpf and in the kidney and spleen of adult fish (de Jong et al. (2005) Annu. Rev. Genet., 39:481-501; Herbomel et al. (1999) Development 126:3735-45). There is rag1 expression and evidence of thymic development by 65-75 hpf, and the thymus is fully mature with medullary and cortical tissues and tcra gene expression by 3 weeks of age (Zapata et al. (2006) Fish Shellfish Immunol., 20:126-36). There is some evidence that B cells first develop in the zebrafish pancreas as evidenced by rag1 transcripts as early as 3-4 dpf (Danilova et al. (2002) PNAS 99):13711-6; Lam et al. (2004) Dev. Comp. Immunol., 28:9-28).

Importantly, zebrafish orthologs have been identified for several known mammalian proto-oncogenes and tumor-suppressor genes involved in leukemogenesis (Kalev-Zylinska et al. (2002) Development 129:2015-30; Kataoka et al. (2000) Mech. Dev., 98:139-43; Lieschke et al. (2002) Dev. Biol., 246:274-95; Schreiber-Agus et al. (1993) Mol. Cell. Biol., 13:2765-75). Gene expression profiling has revealed that several MLL partner genes are represented in the zebrafish genome (Song et al. (2004) PNAS 101:16240-5). Also of relevance to this project are the recently identified functional zebrafish orthologs of mammalian Bcl-2 family members (Kratz et al. (2006) Cell Death Differ., 13:1631-40). The high evolutionary conservation reinforces the notion that zebrafish is a worthwhile model for investigating hematopoiesis and leukemia. By transiently expressing the human AML-associated RUNX1-CBF2T1 fusion oncogene under control of the CMV promoter in zebrafish embryos Kalev-Zylinska et al. reproduced the hematopoietic defects seen in RUNX1-CBF2T1 transgenic mice (Kalev-Zylinska et al. (2002) Development 129:2015-30). A transient TEL-JAK2 fusion oncoprotein transgenic zebrafish also recently was generated (Onnebo et al. (2005) Exp. Hematol., 33:182-8). In addition, Langenau et al. reported the first stable transgenic zebrafish, in which expression of a murine c-Myc-GFP under control of the rag2 promoter induced clonal, transplantable T-cell ALL (Langenau et al. (2003) Science 299):887-90). Notably, an MLL ortholog in Fugu rubripes (pufferfish) with functionally similar domains to its mammalian counterparts has been cloned (Caldas et al. (1998) Oncogene 16:3233-41).

Zebrafish have been extremely powerful for studying hematopoiesis. Many zebrafish orthologs of mammalian hematopoietic genes have been characterized and zebrafish models of leukemia are emerging. The instant invention provides and characterizes the zebrafish ortholog of the human MLL gene.

### SUMMARY OF THE INVENTION

The broad objective of this application is to exploit the zebrafish model to understand the role of human MLL in normal and malignant hematopoiesis. The MLL gene at chromosome band 11q23 is an important oncogene that is disrupted by chromosomal translocations with more than 50 partner genes in infant leukemias and secondary leukemias after chemotherapeutic topoisomerase II poisons. A number of novel MLL partner genes have been identified in human leukemias that predict heterogeneous protein products with diverse functions in variable cellular locations. MLL leukemias have also to be shown to have defective apoptosis regulation. MLL encodes a complex transcription factor that undergoes taspase1 proteolytic cleavage into amino and carboxyl fragments that re-associate in a multiprotein complex and regulate expression of HOX genes, cell cycle genes and other unknown targets. Experiments in mice indicate that MLL is critical for normal hematopoiesis and that the protein product of the der(11) chromosome is leukemogenic.

Zebrafish have become increasingly popular for studying blood cell development because many zebrafish orthologs of blood-specific genes have been identified, and the rapid, external development of abundant, transparent embryos enables real-time functional observations unlike other models. Moreover, transgenic zebrafish models of other leukemias have yielded phenotypes that recapitulate leukemia in humans. The zebrafish mll cDNA is provided herein. Further, mll depletion in zebrafish embryos is shown herein to be associated with blood cell and neuronal defects resembling abnormalities in Mll−/− mice. The embryos are also characterized by small size, a feature of Taspase1 −/− mice. The neuronal defect phenocopies that in zebrafish following runx1 depletion. The observed mll knockdown phenotype in zebrafish embryos is likely a consequence of interplay of mll in pathways that control apoptosis, differentiation, angiogenesis and cell proliferation.

The instant invention encompasses the zebrafish MLL and nucleic acid molecules encoding the same. In a particular embodiment, the zebrafish MLL has at least 90% identity with SEQ ID NO: 2 and nucleic acid molecules encoding the zebrafish MLL have at least 90% identity with SEQ ID NO: 1.

The instant invention also encompasses a zebrafish model of MLL leukemogenesis. In one embodiment, the transgenic zebrafish has reduced expression of zebrafish MLL compared to wild-type. In a particular embodiment, the transgenic zebrafish is zebrafish mll null. In yet another embodiment, the transgenic zebrafish comprises an antisense molecule directed to zebrafish mll.

The zebrafish mll model can be used to examine the effects of enhancing and suppressing normal MLL. Further, the instant invention encompasses transgenic zebrafish comprising Mll linked to specific partner genes. The zebrafish model of the instant invention provides a rapid screening tool to identify anti-cancer agents, particularly anti-leukemia agents.

### BRIEF DESCRIPTIONS OF THE DRAWINGS

FIG. 1A is a schematic of predicted and partially cloned zebrafish mll sequences in Ensembl and GenBank Databases. FIG. 1B is a schematic of a Conserved Domain Architecture Retrieval Tool (CDART) analysis of human and zebrafish MLL proteins depicting conserved domains of human and zebrafish MLL protein fragments. The shown hypothetical zebrafish MLL was obtained by joining of the two zebrafish “similar to MLL” protein sequences are shown.

FIG. 2A provides the amino acid sequences of the highly homologous SET domains from human, mouse, pufferfish (fugu) MLL and Drosophila trx aligned by ClustalW (SEQ ID NOs: 5-8 from top to bottom). Identical amino acid sequences are shown with asterisks. Degenerate primer mixtures for RT-PCR were designed from the highlighted regions. FIG. 2B provides an example of approach to primer design. The provided amino acid sequence is SEQ ID NO: 9 and the nucleotide sequences are SEQ ID NOs: 10-15. FIG. 2C is an image of a gel showing the 203 basepair PCR product with degenerate primer mixtures B and C (first lane) or b and c (third lane).

FIG. 3A is a schematic of the simulated restriction mapping of predicted zebrafish mll genomic sequence. FIG. 3B is an image of an autoradiograph of zebrafish genomic DNAs and normal human subject peripheral blood lymphocyte DNA after probing with B859. A BamHI-digested human DNA is included as a positive control.

FIG. 4 provides a schematic of XM_{—}680024 and XM_{—}679940 and primers used in PCR reactions on zebrafish cDNA. An image of a gel comprising the generated single product that spanned both cDNAs is provided.

FIGS. 5A and 5B are images of gels sowing the PCR amplification of 5′ UTR of zebrafish mll by 5′ RACE and the cloning of 12.4 kb fragment of zebrafish mll cDNA, respectively. FIG. 5C is a schematic of the 5′ UTR and 35-exon overlapping sequences generated in FIG. 5A and FIG. 5B, which together contain near complete zebrafish mll cDNA.

FIG. 6A is a ClustalW alignment of human MLL protein sequence (GenBank accession no. AAA58669; SEQ ID NO: 22) and sequence of predicted zebrafish mll protein (SEQ ID NO: 23) derived by assembling the cloned 12412 bp fragment, the 5′ coding sequence in zebrafish mll cDNA, and the 3′ 46 bases taken from Entrez Gene 557048. Shaded regions indicate protein domains that both species have conserved. FIG. 6B is a schematic of the protein domain alignment. Percent amino acid sequence identity is indicated. DGVDD (SEQ ID NO: 24) and DGADD (SEQ ID NO: 25) cleavage sites are shown.

FIG. 7 is an image of a Northern blot (top) and a corresponding ethidium-stained gel (bottom) of total zebrafish RNA collected at the indicated times. For the Northern blot, the 12.4 kb fragment of zebrafish mll cDNA was used as a probe is at bottom.

FIG. 8 provides images of gels comprising RT-PCR products of zebrafish mll mRNA expression. Primers used for the indicated region are also provided (forward and reverse primers are SEQ ID NOs: 26-29 and SEQ ID NOs: 30-33, top to bottom, respectively).

FIG. 9 is a graph of the quantitative RT-PCR analysis of temporal expression of zebrafish mll mRNA expression in wild type zebrafish embryos and whole wild type adult. The dark grey bars compare the normalized zebrafish mll expression to the normalized zebrafish mll expression in the adult. The light grey bars represent the 2^{-ΔΔCT} analysis of the relative changes in zebrafish mll expression as a function of the age of the embryo compared to the adult with expression in the adult calibrator sample set to one.

FIG. 10 is a graph of the quantitative RT-PCR analysis of tissue specific expression zebrafish mll mRNA expression in wild type adult zebrafish. The relative abundance of zebrafish mll mRNA in the indicated tissues was compared to zebrafish mll mRNA expression in the whole adult by analysis of absolute copy number from the standard curves (dark grey bars) and by analysis of relative gene expression by the 2^{-ΔΔCT} method (light grey bars).

FIG. 11A is a schematic of the zebrafish mll exon 2-intron 2 splice-site targeted morpholino construct (MO E2I2). Grey lines indicate normal transcript splicing and black lines indicate aberrant splicing of exon 1 to exon 3. The thick black line indicates a second form of aberrant splicing due to failure to splice out intron 2. FIG. 11B is an image of a gel showing the disruption of zebrafish mll transcript splicing by RT-PCR. The detected products are depicted in the schematics to the right of the gel image. FIG. 11C provides differential interference contrast (DIC) images of zebrafish embryos after mll depletion. Grey arrows indicate aberrant head protrusion and enlarged hindbrain ventricle, black arrow indicates erythroid cells in heart/ventral anterior yolk sac of control, and unfilled black arrows indicate barely visible erythroid cells in morphant.

FIG. 12A provides the 561 bp 5′ RACE sequence (SEQ ID NO: 3) generated from adult zebrafish. The highlighted sequence is the 5′ untranslated region (UTR; SEQ ID NO: 4). FIGS. 12B-12F provide a nucleotide sequence of zebrafish MLL (SEQ ID NO: 1).

FIG. 13 provides the amino acid sequence of zebrafish MLL (SEQ ID NO: 2).

### DETAILED DESCRIPTION OF THE INVENTION

A broad objective of this application is to exploit the zebrafish model to understand the role of human MLL in normal and malignant hematopoiesis. The MLL gene at chromosome band 11q23 is an important oncogene that is disrupted by chromosomal translocations with more than 50 partner genes in infant leukemias and secondary leukemias after chemotherapeutic topoisomerase II poisons (see, e.g., U.S. patent application Ser. Nos. 11/199,544; 10/118,783; and 11/222,626 and U.S. Pat. No. 6,368,791). A number of MLL partner genes in human leukemias have been discovered that predict heterogeneous protein products with diverse functions in variable cellular locations. MLL leukemias have also been shown to have defective apoptosis regulation. MLL encodes a complex transcription factor that undergoes taspase 1 proteolytic cleavage into amino and carboxyl fragments that re-associate in a multiprotein complex and regulate expression of HOX genes, cell cycle genes and other unknown targets. Experiments in mice indicate that MLL is critical for normal hematopoiesis and that the protein product of the der(11) chromosome is leukemogenic. Zebrafish have become increasingly popular for studying blood cell development because many zebrafish orthologs of blood-specific genes have been identified, and the rapid, external development of abundant, transparent embryos enables real-time functional observations unlike other models. Moreover, transgenic zebrafish models of other leukemias have yielded phenotypes that recapitulate leukemia in humans. The zebrafish mll cDNA has been cloned herein and it is shown that mll depletion in zebrafish embryos is associated with blood cell and neuronal defects resembling abnormalities in mll−/− mice. The embryos are also characterized by small size, a feature of Taspase 1 −/− mice. Furthermore, the neuronal defect phenocopies that in zebrafish following runx1 depletion. The functions of MLL in normal blood cell development and leukemia are incompletely understood. However, the mll knockdown phenotype that is observed in zebrafish embryos may be a consequence of interplay of mll in pathways that control apoptosis, differentiation, angiogenesis and cell proliferation. In addition, understanding how wild type mll modulates these processes and how particular partner proteins function in the zebrafish model will provide new inroads to understand the consequences of the translocations.

The zebrafish system of the instant invention allows for the deciphering of the role of mll in zebrafish embryogenesis, determination of its place in zebrafish blood cell development, and provides a prototype to address the role of different human MLL translocations in leukemogenesis. Leukemias with MLL translocations are refractory to current treatments. The use of combinations of approaches to knockdown, over-express and mutate mll in reverse genetic screens will show allow for the determination of the gene network that MLL affects. Further, placement of mll into novel molecular and cellular pathways in zebrafish will provide a rapid screening tool to test anti-leukemic agents targeting MLL fusion proteins or their downstream effectors or interacting pathways.

### I. Definitions

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

A “replicon” is any genetic element, for example, a plasmid, cosmid, bacmid, plastid, phage or virus, which is capable of replication largely under its own control. A replicon may be either RNA or DNA and may be single or double stranded. Generally, a “viral replicon” is a replicon which contains the complete genome of the virus. A “sub-genomic replicon” refers to a viral replicon that contains something less than the full viral genome, but is still capable of replicating itself. For example, a sub-genomic replicon may contain most of the genes encoding for the non-structural proteins of the virus, but not most of the genes encoding for the structural proteins.

A “vector” is a replicon, such as a plasmid, cosmid, bacmid, phage or virus, to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The terms “percent similarity”, “percent identity” and “percent homology” when referring to a particular sequence are used as set forth in the University of Wisconsin GCG software program.

The term “substantially pure” refers to a preparation comprising at least 50-60% by weight of a given material (e.g., nucleic acid, oligonucleotide, protein, etc.). More preferably, the preparation comprises at least 75% by weight, and most preferably 90-95% by weight of the given compound. Purity is measured by methods appropriate for the given compound (e.g. chromatographic methods, agarose or polyacrylamide gel electrophoresis, HPLC analysis, and the like).

The term “oligonucleotide” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and use of the method. For example, for diagnostic applications, depending on the complexity of the target sequence, the oligonucleotide probe typically contains 15-25 or more nucleotides, although it may contain fewer nucleotides. The probes herein are selected to be complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

Polymerase chain reaction (PCR) has been described in U.S. Pat. Nos. 4,683,195, 4,800,195, and 4,965,188, the entire disclosures of which are incorporated by reference herein.

With respect to single stranded nucleic acids, particularly oligonucleotides, the term “specifically hybridizing” refers to the association between two single-stranded nucleotide molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA molecule of the invention, to the substantial exclusion of hybridization of the oligonucleotide with single-stranded nucleic acids of non-complementary sequence. Appropriate conditions enabling specific hybridization of single stranded nucleic acid molecules of varying complementarity are well known in the art.

For instance, one common formula for calculating the stringency conditions required to achieve hybridization between nucleic acid molecules of a specified sequence homology is set forth below (Sambrook et al., 1989, Molecular Cloning: A Laboratory Manual, Cold Spring Harbor Laboratory Press):

Tₘ=81.5° C.+16.6 Log [Na+]+0.41(%G+C)−0.63(%formamide)−600/#bp in duplex

As an illustration of the above formula, using [Na+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The term “gene” refers to a nucleic acid comprising an open reading frame encoding a polypeptide, including both exon and (optionally) intron sequences. The nucleic acid may also optionally include non-coding sequences such as promoter or enhancer sequences. The term “intron” refers to a DNA sequence present in a given gene that is not translated into protein and is generally found between exons.

The term “compound” can be, but is not limited to, a chemical, a small molecule, a drug, an antibody, a peptide, a secreted protein, and a nucleic acid molecule (such as DNA, RNA, a polynucleotide, an oligonucleotide, an antisense molecule, an siRNA, and the like).

As used herein, the term “zebrafish” may refer to any fish or strain of fish that is considered to be of the genus and species, Danio rerio.

The term “transgenic” may refer to an organism and the progeny of such an organism that contains a nucleic acid molecule that has been artificially introduced into the organism.

### II. Nucleic Acid Molecules

Nucleic acid molecules encoding the zebrafish MLL proteins of the invention may be prepared by three general methods: (1) synthesis from appropriate nucleotide triphosphates, (2) isolation from biological sources, and (3) mutation of nucleic acid molecule encoding zebrafish MLL proteins. These methods utilize protocols well known in the art. The availability of nucleotide sequence information, such as the sequences provided herein, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be purified according to methods known in the art, such as high performance liquid chromatography (HPLC). Long, double-stranded polynucleotides may be synthesized in stages, due to any size limitations inherent in the oligonucleotide synthetic methods.

Nucleic acid sequences encoding the zebrafish MLL proteins of the invention may be isolated from appropriate biological sources using methods known in the art. In one embodiment, a cDNA clone is isolated from a cDNA expression library of human origin. In an alternative embodiment, utilizing the sequence information provided by the cDNA sequence, human genomic clones encoding zebrafish MLL proteins may be isolated. Additionally, cDNA or genomic clones having homology with zebrafish MLL may be isolated from other species using oligonucleotide probes corresponding to predetermined sequences within the zebrafish MLL encoding nucleic acids.

Exemplary nucleotide sequences encoding the zebrafish MLL proteins are provided hereinbelow. A zebrafish MLL nucleotide sequence may have 75%, 80%, 85%, 90%, 95%, 97%, or 99% homology with SEQ ID NO: 1. The 5′UTR (SEQ ID NO: 4) may also be included at the 5′ end of the zebrafish MLL encoding nucleic acid molecule.

In accordance with the present invention, nucleic acids having the appropriate level of sequence homology with a nucleic acid molecule encoding the zebrafish MLL proteins may be identified by using hybridization and washing conditions of appropriate stringency.

Nucleic acids of the present invention may be maintained as DNA in any convenient vector. The zebrafish MLL encoding nucleic acid molecule may be linked to at least one expression operon. Zebrafish MLL encoding nucleic acid molecules of the invention include cDNA, genomic DNA, RNA, and fragments thereof which may be single- or double-stranded. Thus, this invention provides oligonucleotides having sequences capable of hybridizing with at least one sequence of a nucleic acid molecule of the present invention.

Also contemplated in the scope of the present invention are oligonucleotide probes which specifically hybridize with the zebrafish MLL nucleic acid molecules of the invention under high or very high stringency conditions. Primers capable of specifically amplifying zebrafish MLL encoding nucleic acids described herein are also contemplated herein. As mentioned previously, such oligonucleotides are useful as probes and primers for detecting, isolating or amplifying zebrafish MLL genes.

It will be appreciated by persons skilled in the art that variants (e.g., allelic variants) of zebrafish MLL sequences exist, and must be taken into account when designing and/or utilizing oligonucleotides of the invention. Accordingly, it is within the scope of the present invention to encompass such variants, with respect to the zebrafish MLL sequences disclosed herein or the oligonucleotides targeted to specific locations on the respective genes or RNA transcripts. Accordingly, the term “natural allelic variants” is used herein to refer to various specific nucleotide sequences of the invention and variants thereof that would. The usage of different wobble codons and genetic polymorphisms which give rise to conservative or neutral amino acid substitutions in the encoded protein are examples of such variants. Additionally, the term “substantially complementary” refers to oligonucleotide sequences that may not be perfectly matched to a target sequence, but such mismatches do not materially affect the ability of the oligonucleotide to hybridize with its target sequence under the conditions described.

The present invention also encompasses antisense nucleic acid molecules which may be targeted, for example, to translation initiation sites and/or splice sites to inhibit the expression of zebrafish mll. Such antisense molecules are typically between about 15 and about 30 nucleotides in length. Antisense constructs may also be generated which contain the entire zebrafish mll sequence in reverse orientation. Antisense oligonucleotides targeted to any known nucleotide sequence can be prepared by oligonucleotide synthesis according to standard methods.

Small interfering RNA (siRNA) molecules designed to inhibit expression of IDO2 are also encompassed in the instant invention. Typically, siRNA molecules are double stranded RNA molecules between about 12 and 30 nucleotides in length, more typically about 21 nucleotides in length (see Ausubel et al., eds. Current Protocols in Molecular Biology, John Wiley and Sons, Inc., (2005)).

Several methods of modifying oligonucleotides are known in the art. For example, methylphosphonate oligonucleotide analogs may be synthesized wherein the negative charge on the inter-nucleotide phosphate bridge is eliminated by replacing the negatively charged phosphate oxygen with a methyl group (see Uhlmann et al., Chemical Review, 90: 544-584 (1990)). Another common modification is the synthesis of oligodeoxyribonucleotide phosphorothioates. In these analogs, one of the phosphate oxygen atoms not involved in the phosphate bridge is replaced by a sulphur atom, resulting in the negative charge being distributed asymmetrically and located mainly on the sulphur atoms. When compared to unmodified oligonucleotides, oligonucleotide phosphorothioates are improved with respect to stability to nucleases, retention of solubility in water and stability to base-catalyzed hydrolysis (see Uhlmann et al., supra at 548-50; Cohen, J. S. (ed.) Oligodeoxynucleotides: Antisense Inhibitors of Gene Expression, CRC Press, Inc., Boca Raton, Fla. (1989)). These references also provide other modifications of oligonucleotides.

In a particular embodiment of the instant invention, the oligonucleotides are modified with morpholine rings. A morpholino oligonucleotides comprises morpholine rings replacing the ribose or deoxyribose sugar moieties and non-ionic phosphorodiamidate linkages replacing the anionic phosphates of DNA and RNA. Each morpholine ring suitably positions one of the standard bases. Notably, the backbone of a morpholino oligonucleotide is not recognized by cellular enzymes. Accordingly, these oligonucleotides are stable against nucleases.

Still other modifications of the oligonucleotides may include coupling sequences that code for RNase H to the antisense oligonucleotide. This enzyme (RNase H) will then hydrolyze the hybrid formed by the oligonucleotide and the specific targeted mRNA. Alkylating derivatives of oligonucleotides and derivatives containing lipophilic groups can also be used. Alkylating derivatives form covalent bonds with the mRNA, thereby inhibiting their ability to translate proteins. Lipophilic derivatives of oligonucleotides will increase their membrane permeability, thus enhancing penetration into tissue. Besides targeting the mRNAs, other antisense molecules can target the DNA, forming triple DNA helixes (DNA triplexes). Another strategy is to administer sense DNA strands which will bind to specific regulator cis or trans active protein elements on the DNA molecule.

Deoxynucleotide dithioates (phosphorodithioate DNA) may also be utilized in this invention. These compounds which have nucleoside-OPS20 nucleoside linkages, are phosphorus achiral, anionic and are similar to natural DNA. They form duplexes with unmodified complementary DNA. They also activate RNase H and are resistant to nucleases, making them potentially useful as therapeutic agents. One such compound has been shown to inhibit HIV-1 reverse transcriptase (Caruthers et al., INSERM/NIH Conference on Antisense Oligonucleotides and Ribonuclease H, Arcachon, France 1992). In accordance with the present invention, antisense oligonucleotides and siRNA may be delivered directly or may be produced by expression of DNA sequences cloned into plasmid or retroviral vectors. Using standard methodology known to those skilled in the art, it is possible to maintain the antisense RNA-encoding DNA in any convenient cloning vector (see Ausubel et al., eds. Current Protocols in Molecular Biology, John Wiley and Sons, Inc., (2005)).

Various genetic regulatory control elements may be incorporated into antisense RNA-encoding expression vectors to facilitate propagation in both eukaryotic and prokaryotic cells. Different promoters may be utilized to drive expression of the antisense sequences, the cytomegalovirus immediate early promoter being preferred as it promotes a high level of expression of downstream sequences. Polyadenylation signal sequences are also utilized to promote mRNA stability. Sequences preferred for use in the invention include, but are not limited to, bovine growth hormone polyadenylation signal sequences or thymidine kinase polyadenylation signal sequences. Antibiotic resistance markers are also included in these vectors to enable selection of transformed cells. These may include, for example, genes that confer hygromycin, neomycin or ampicillin resistance.

Transgenic animals and cells are also encompassed by the instant invention. The term “transgenic animal” is intended to include any non-human animal, preferably vertebrate, in which one or more of the cells of the animal contain at least one heterologous or foreign nucleic acid molecule. Non-human animals include, without limitation, rodents, non-human primates, sheep, dog, cow, amphibians, fish (e.g, zebrafish, medaka, and the like), and reptiles. In a preferred embodiment, the animal is a zebrafish.

Mll knockout animals are also encompassed by the instant invention. Modifications, insertions, and/or deletions may render the naturally occurring gene nonfunctional, thereby producing a “knock out” transgenic animal (e.g., zebrafish mll^{-/-}). For example, retroviral insertion may be used to render zebrafish mll nonfunctional (e.g., reduce or eliminate production of mll). Alternatively, the naturally occurring gene may be rendered nonfunctional by introducing an siRNA or an antisense molecule (e.g., a morpholino antisense molecule) directed at mll. Transgenic animals of the instant invention are useful as a nonhuman model for diseases involving mll. The transgenic animals may also be used as in vivo models for drug screening studies for certain human diseases, and for eventual treatment of disorders or diseases associated with mll.

The instant invention also encompasses transgenic animals comprising a heterologous nucleic acid encoding an MLL translocation. The MLL translocation can be a human MLL translocation (see, e.g., U.S. patent application Ser. Nos. 11/199,544; 10/118,783; and 11/222,626 and U.S. Pat. No. 6,368,791). Additionally, the MLL translocation can be a zebrafish translocation, particularly one that corresponds to a human MLL translocation. Indeed, as described hereinbelow, MLL partner gene analogs have been identified in zebrafish. As such, translocations of the zebrafish analogs of the genes involved in the human MLL translocation may be generated and expressed in zebrafish. In a particular embodiment, one allele of the transgenic animal is wild-type mll and the other allele is an mll translocation.

In a particular aspect, the transgenic fish may be generated by introducing a heterologous nucleic acid molecule into a fish egg cell or embryonic cell. The heterologous nucleic acid molecule may comprise an expression vector. The heterologous nucleic acid molecule may be expressed only transiently in the fish or may be stably integrated into the genome of the injected cell. The heterologous nucleic acid may be transmitted to the progeny of the transgenic fish. Notably, Fan et al. have demonstrated homologous recombination in zebrafish embryonic stem cells (Transgenic Res. (2006) 15:21-30).

In yet another embodiment, the transgenic animals of the instant invention may express mll from another species and/or may over-express zebrafish mll. Additionally, the transgenic animal may express mll linked to a partner gene, such as those described hereinbelow.

Transgenic zebrafish and methods of producing the same are described in U.S. Pat. No. 6,953,875 and U.S. Patent Application Publication Nos. 20050120392, 20040261143, 20040143865, 20020178461, 20040117867, and 20020187921.

The instant invention also encompasses cells isolated from the transgenic animals. In a particular embodiment, the cells are phagocytic B cells and/or precursors thereof (see, e.g., Li et al. (2006) Nat. Immunol., 7:1116-24).

### III. Proteins

Zebrafish MLL proteins of the present invention maybe prepared in a variety of ways, according to known methods. The proteins may be purified from appropriate sources, e.g., transformed bacterial or animal cultured cells or tissues, by immunoaffinity purification. The availability of nucleic acid molecules encoding zebrafish MLL proteins enables production of the proteins using in vitro expression methods and cell-free expression systems known in the art. In vitro transcription and translation systems are commercially available, e.g., from Promega Biotech (Madison, Wis.) or Gibco-BRL (Gaithersburg, Md.).

Alternatively, larger quantities of zebrafish MLL proteins may be produced by expression in a suitable prokaryotic or eukaryotic system. For example, part or all of a DNA molecule encoding for zebrafish MLL proteins may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

Zebrafish MLL proteins produced by gene expression in a recombinant procaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemagglutinin epitope. Such methods are commonly used by skilled practitioners.

Zebrafish MLL proteins of the invention, prepared by the aforementioned methods, may be analyzed according to standard procedures. For example, such protein may be subjected to amino acid sequence analysis, according to known methods.

Exemplary amino acid sequences of zebrafish MLL proteins are provided hereinbelow. Zebrafish MLL amino acid sequence may have 75%, 80%, 85%, 90%, 95%, 97%, or 99% homology with SEQ ID NO: 2.

The present invention also encompasses antibodies capable of immunospecifically binding to proteins of the invention. Polyclonal antibodies directed toward zebrafish MLL proteins may be prepared according to standard methods. In a preferred embodiment, monoclonal antibodies are prepared, which react immunospecifically with the various epitopes of the zebrafish MLL proteins. Monoclonal antibodies may be prepared according to general methods known in the art. Polyclonal or monoclonal antibodies that immunospecifically interact with zebrafish MLL proteins can be utilized for identifying and purifying such proteins. For example, antibodies may be utilized for affinity separation of proteins with which they immunospecifically interact. Antibodies may also be used to immunoprecipitate proteins from a sample containing a mixture of proteins and other biological molecules.

### IV. MLL Partner Genes

Various panhandle PCR approaches have been developed for characterizing MLL translocations (Raffini et al. (2002) PNAS, 99:4568-73; Megonigal et al. (2000) PNAS, 97:2814-9; Megonigal et al. (1998) PNAS, 95:6413-8; Megonigal et al. (1997) PNAS, 94:11583-8; Felix et al. (1997) Blood, 90:4679-86; Felix et al. (1998) Leukemia, 12:976-81; Megonigal et al. (2000) PNAS, 97:9597-602; Robinson et al. (2006) Genes Chromosomes Cancer, 45:740-53), have been central to unraveling the partner genes of MLL and linking different partner genes to disease and patient features. In studying >80 de novo and treatment-related leukemias, the new MLL partner genes shown in Table 1 have been discovered. hCDCrel, which is a member of the SEPTIN family, was found to be fused to MLL in identical, non-constitutional t(11;22)(q23;q11.2) translocations in AML of infant twins (Megonigal et al. (1998) PNAS, 95:6413-8). Several of the partner genes were discovered in complex, three-way rearrangements. Another SEPTIN family member SEPTIN6, has been identified as a partner gene of MLL in a case of infant AML with a complex t(3;X;11) rearrangement (Slater et al. (2002) Oncogene, 21:4706-14). The karyotype in a case of infant AML suggested t(3;11) (q29;q23) but panhandle PCR identified a fusion of MLL to the MYO1F gene from band 19p13, unmasking another complex rearrangement. CDK6, the first cell cycle regulatory gene fused to MLL, was found in a 5′-CDK6-MLL-3′ breakpoint junction of a complex translocation in a case of infant ALL (Raffini et al. (2002) PNAS, 99:4568-73). The term ‘partner gene’ generally refers to the gene whose 3′ sequence is fused to the 5′ sequence of MLL but here the 3′ sequence of MLL was fused to the 5′ sequence of CDK6, and an in-frame 5′-CDK6-MLL-3′ transcript was produced in addition to a 5′-MLL-AF4-3′ transcript (Raffini et al. (2002) PNAS, 99:4568-73). Most recently a cryptic, complex three-way MLL, AF10, ARMC3 translocation was identified in a case of secondary AML generated a 5′-ARMC3-MLL-3′ breakpoint junction and the corresponding transcript. The uncharacterized ARMC3 protein contains Arm repeats similar to catenin family proteins implicated in leukemia (Jamieson et akl. (2004) N. Engl. J. Med., 351:657-67) and cancer (Brembeck et al. (2006) Curr. Opin. Genet. Dev., 16:51-9). In a case of infant AML, the ribosomal protein S3 (RPS3) gene from chromosome band 11q13.3-11q13.5, the gene product of which regulates initiation of translation, was discovered at the 5′-RPS3-MLL-3′ junction of a three-way MLL, AF10, RPS3 rearrangement. alkaline ceramidase is a new partner gene of MLL at band 19p13. The unusual finding that the der(11) and der(19) breakpoints in this partner gene were both in the 3′ UTR predicted a truncated MLL der(11) protein product and, conversely, a der(19) protein product with the entire alkaline ceramidase protein fused to the MLL C terminus. Thus, not only are there many partner genes, but also they are involved in heterogeneous types of rearrangements.

Many MLL partner genes encode important proteins in transcriptional regulation or signaling pathways in different cellular compartments (Ayton et al. (2003) Genes Dev., 17:2298-307), but the significance of their disruption in MLL leukemogenesis is not well understood. In addition, although murine studies clearly demonstrate that the der(11) (i.e. 5′-partner-MLL-3′) gene product is leukemogenic (Ayton et al. (2003) Genes Dev., 17:2298-307), the nature of these partner genes raises questions about whether disruption of the partner protein by the 5′-partner-MLL-3′ rearrangement may be a critical second hit (He et al. (2000) Mol. Cell., 6:1131-41) in cases where 5′-partner-MLL-3′ transcripts are produced.

An MLL-GAS7 translocation that was discovered was associated with a highly aggressive secondary AML after a short latency from the primary cancer treatment (Megonigal et al. (2000) PNAS, 97:2814-9). In contrast, a patient was prospectively followed with primary neuroblastoma whose marrow was completely replaced with a clone harboring a highly novel MLL translocation with the FRYL gene from chromosome band 4p12 without any clinical evidence of leukemia until beyond the typical latency when secondary MDS was diagnosed. The FRYL protein is homologous to Fry (gene name furry), which regulates bristle morphogenesis in Drosophila. This partner gene is of further interest because infant, pediatric, and adult leukemia subsets without this translocation express high levels of FRYL RNA. A second patient currently is nine years from detection of an MLL rearrangement in the marrow that subsequently regressed without any evidence of disease; the partner gene associated with this clinical behavior encodes the Notch co-activator MAML2.

Thus the many partner genes of MLL result in a heterogeneous spectrum of diseases with variable clinical behaviors, phenotypic and morphologic characteristics. Notably, a search of the NCBI and other databases indicates that homologues of many of the partner genes that were discovered can be found in zebrafish (Table 1).

TABLE 1
MLL Partner Genes

| | | | | Zebrafish |
|---|---|---|---|---|
| Gene | Location | Protein | Leukemia | Homologue |
| GMPS | 3q24 | amidotransferase | t-AML | NP_956881 |
| CDK6 | 7q21 | kinase | ALL | XP_698003 |
| RPS3 | 11q13-q15 | ribosomal protein | AML | AAQ94564 |
| GAS7 | 17p13 | transcription factor, | t-AML | ENSDARP00000077209 |
| | | synapsin, W-W motifs | | |
| MYO1F | 19p13 | myosin family | AML | XP_693434 |
| ALKALINE | 19p13 | ceramidase | ALL | Q56812 |
| CERAMIDASE | | | | |
| hCDCrel | 22q11.2 | septin family | AML | AAH78256 |
| SEPTIN6 | Xq23 | septin family | AML | NP_997791 |
| MAML2 | 11q21 | transcriptional coactivator | none | |
| FRY-L | 4p12 | ? | t-MDS | XP_686711 |
| ARMC3 | 10p12 | ARM repeats | t-AML | XP_688618 |
| ACTN4 | 19q13 | spectrin family | ALL | NP_955880 |
| RYR1 | 19q13 | ryanodine receptor | ALL | XP_694415 |
| KIAA0999 | 11q23 | ? | t-AML | AAH70022 |

Since impaired apoptosis is an avenue to chemotherapy resistance (Reed, J C (2003) Cancer Cell, 3:17-22), a characteristic feature of infant leukemias with MLL translocations (Pieters et al. (1998) Leukemia, 12:1344-8; Pui et al. (2002) Lancet, 359:1909-15), a custom, high-throughput TaqMan array was employed to compare expression patterns of cell death/survival genes in the diagnostic bone marrow specimens from 89 primary pediatric leukemia cases (85/89 infant leukemia; 61 ALL, 28 AML; 30 t(4;11), 26 other MLL rearrangement, 33 MLL rearrangement negative), the ALL cell lines RS4:11 and SEM-K2, and the AML cell line MV4-11. BCL-2 mRNA expression normalized to ACTB and relative to normal CD34+ cells was compared in leukemia cases classified by MLL rearrangement status. Relative expression values were determined using the 2^{-ΔΔCT} method. Relative BCL-2 mRNA expression levels in lineage subtypes show a significant difference between ALL vs. AML.

Regression tree models were constructed to examine the ability of disease and patient-specific predictors to explain the variation in BCL-2 mRNA expression. The results of these experiments indicate that increased anti-apoptotic BCL-2 expression is characteristic of many cases of MLL-rearranged acute leukemia in infants, and that high BCL-2 expression distinguishes cases with t(4;11) from cases with other MLL translocations, which further segregate from cases without MLL rearrangements. Interestingly, as early as 1998 Yu et al. demonstrated increased TUNEL staining and implicated increased apoptosis in the branchial arch hypoplasia in Mll^{-/-} embryos (Yu et al. (1998) PNAS, 95:10632-6).

### V. Zebrafish MLL

As stated herein, microscopic observations reveal that the depletion of zebrafish mll during early embryogenesis grossly recapitulates the neuronal and hematopoietic defects of Mll^{-/-} mice, the small size of Taspase1^{-/-} mice, and the hindbrain abnormality of zebrafish runx1 morphants. Related studies on the human disease showed that the human MLL gene undergoes chromosomal translocations with a considerable number of partner genes, many of which encode proteins in critical pathways in the cell. Additional studies showed that infant leukemias with MLL translocations exhibit high levels BCL-2 mRNA expression. On the basis of these studies, MLL and MLL fusion proteins have broad and novel functions in the cell relating to apoptosis, differentiation, angiogenesis and proliferation.

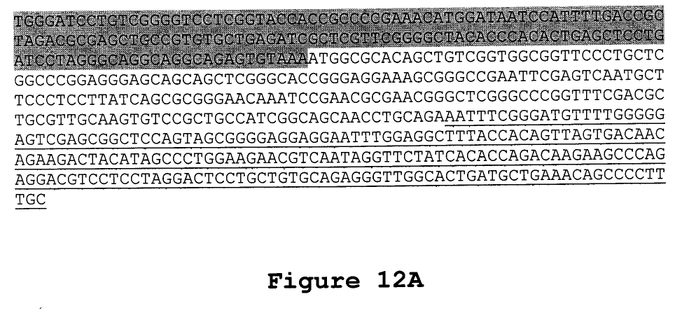

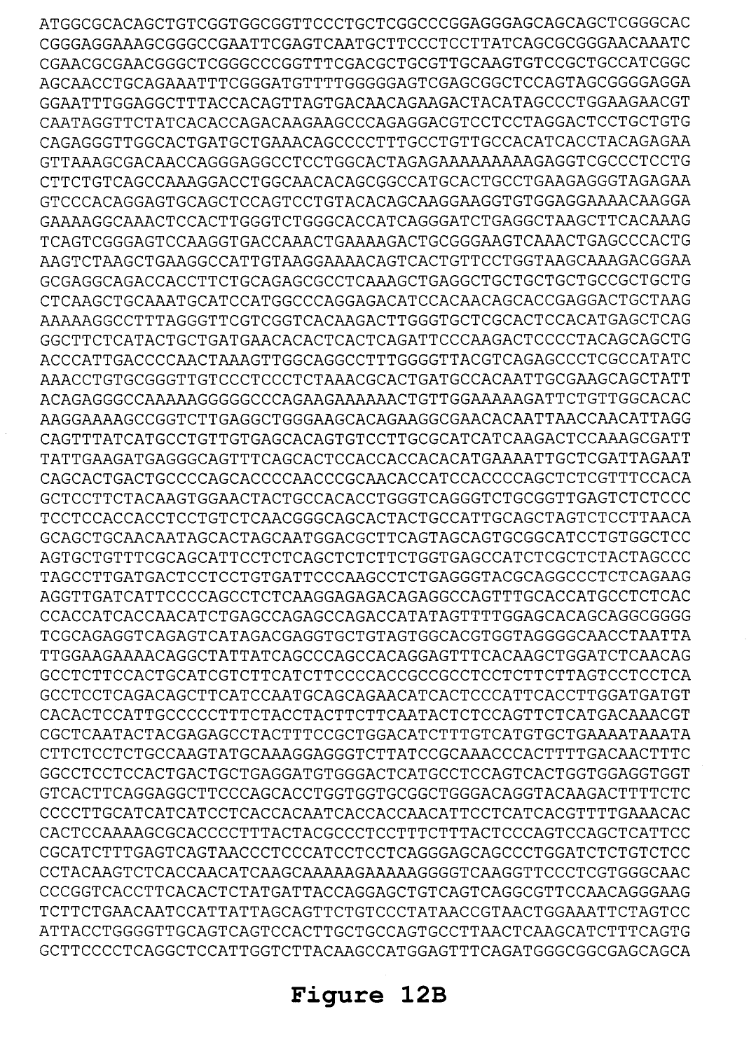

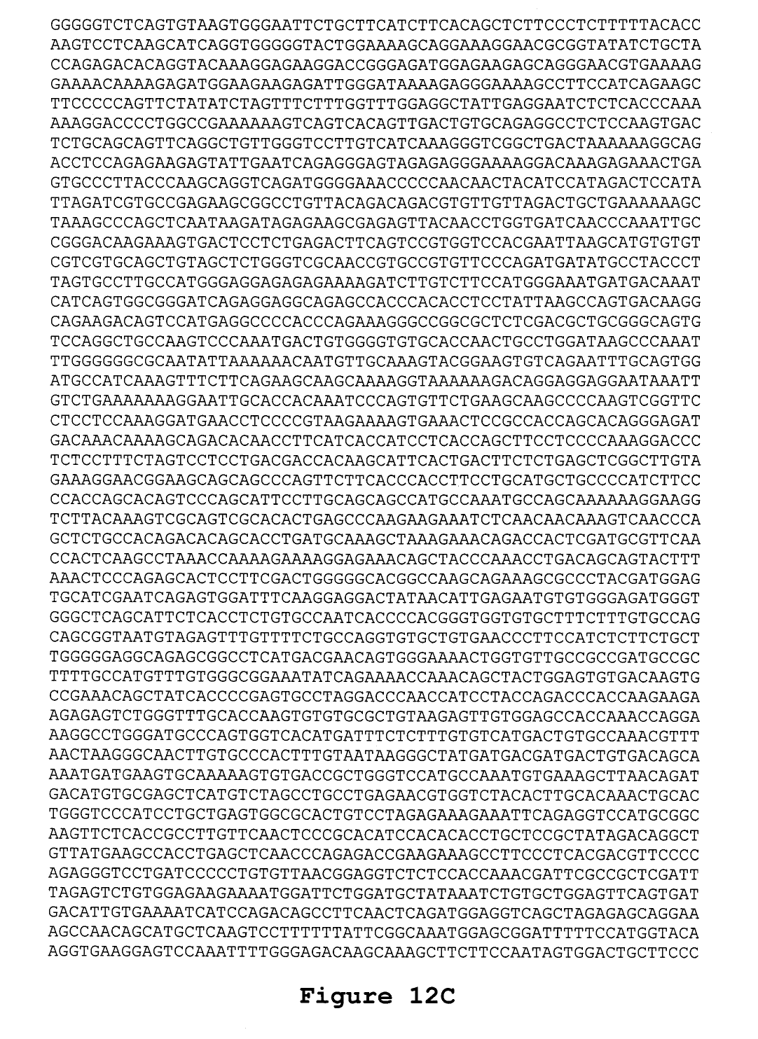

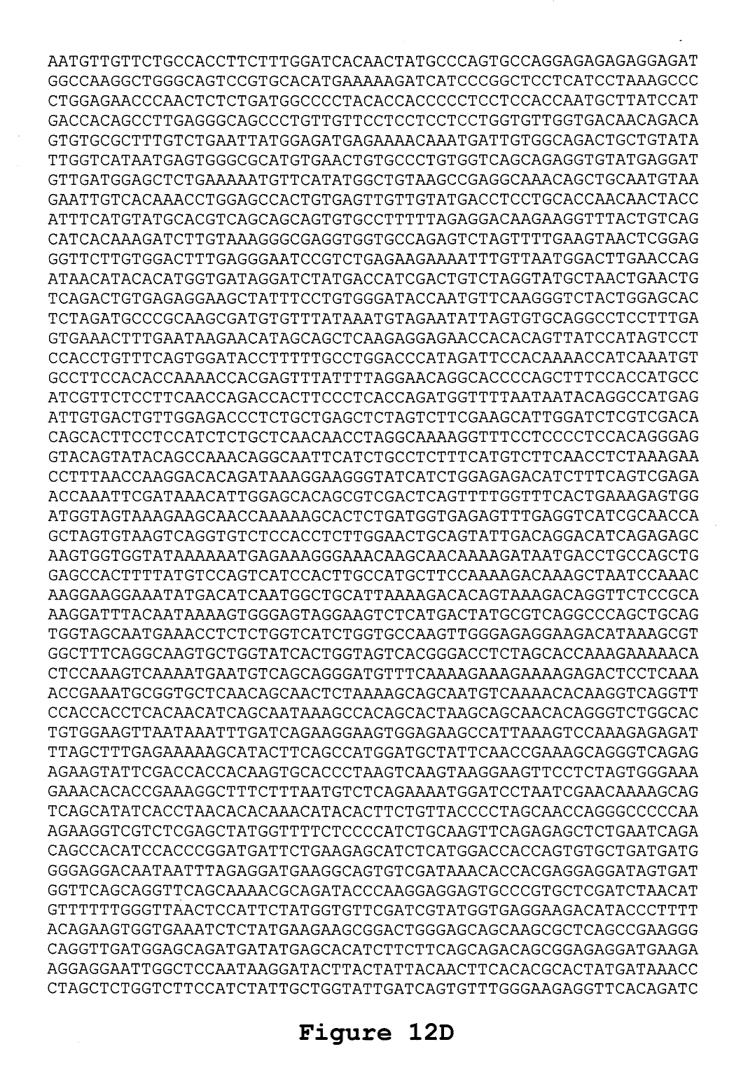

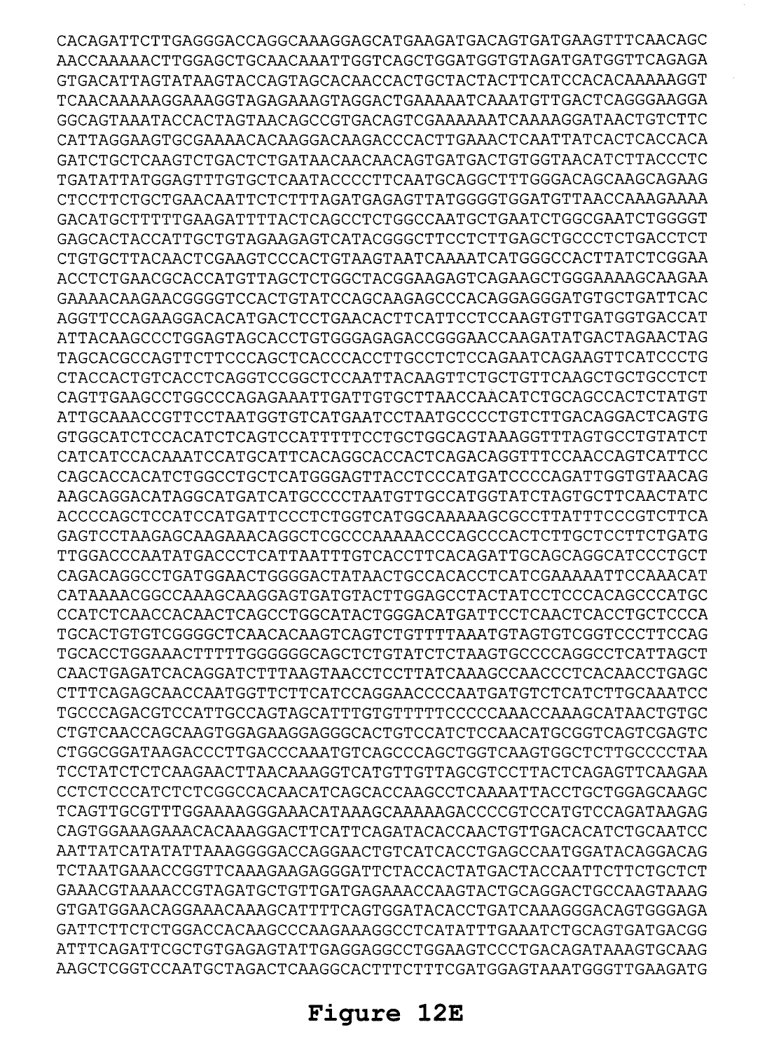

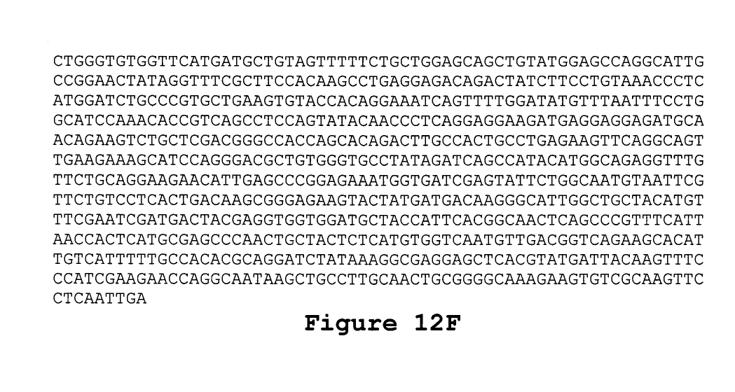

Herein, the zebrafish ortholog of human MLL has been cloned and characterized. This allows for the use of the zebrafish system to model the cellular functions controlled by the normal zebrafish mll and dysregulated by MLL fusion transgenes. As described below, a 12657 bp nucleic acid molecule encoding zebrafish mll is provided (FIGS. 12B-F).

The temporal pattern of zebrafish mll RNA expression in whole wild-type zebrafish embryos and adults has been examined hereinbelow. The detection of an intense signal at the level of the less sensitive Northern blot at 2 hpf, which is a timepoint before zygotic transcripts are produced (Chatterjee et al. (2005) Dev Dyn., 233:890-906; Christie et al. (2004) Am. J. Physiol. Heart Circ. Physiol., 286:H1623-32), indicates that abundant maternal zebrafish mll transcripts are supplied to the embryo. RT-PCR analysis at 2 hpf and the other timepoints, all later than 5 hpf when maternal transcripts are degraded, indicated that zygotic mll is expressed throughout embryogenesis and into the adult. In addition the less sensitive Northern blot detected expression of zygotic mll expression by 24 hpf and at the subsequent timepoints.

Notably, the temporal pattern of zebrafish mll expression in specific tissues can also be characterized. Quantitative RT-PCR (qRT-PCR) and Northern blot analysis may be performed on pooled blood cells isolated by cardiac puncture (Craven et al. (2005) Blood 105:3528-34) at sequential times after 24 hpf, i.e. when blood cells become visible in the circulation. The kidney marrow, spleen, liver, pancreas as well as non-hematopoietic tissues (e.g. brain, eye, muscle, bone) may also be dissected upon becoming visible and at sequential timepoints forward. RNAs from these respective sources may be TRIzol extracted, DNAse treated and, where necessary, pooled for the analyses. The sensitivity of the Northern blot analysis may be augmented by using poly-A+ RNA. For the qRT-PCR analysis, a high throughput TaqMan low density array may be utilized similar to that described hereinabove. Several amplicons within zebrafish mll may be amplified using primers crossing exon boundaries. The results of qRT-PCR may be verified by RT-PCR with gene specific primers and sequencing of the products.

The spatio-temporal patterns of zebrafish mll mRNA expression may also be examined by whole-mount in situ hybridization (WISH) analysis performed on whole-mounted fish. A digoxygenin-labeled mll RNA antisense probe may obtained by reverse transcribing the zebrafish mll cDNA, which already has been generated, and the probe may be used in time course assays from the single cell stage through adult. Standard protocols may be followed for embryo dechlorination and fixation, embryonic pigment removal after 24 hpf, embryo bleaching, hybridization of the probe and antibody detection of the signal (Paffett-Lugassy et al. (2005) Methods Mol. Med., 105:171-98).

There are complementary strategies which may be undertaken to characterize the temporal expression of zebrafish mll in specific blood cell lineages. The first two strategies involve analyses of hematopoietic cell subsets from the blood cells collected from the heart and hematopoietic tissues. The cells from the tissues may be disaggregated by passage through a filter (Rhodes et al. (2005) Dev. Cell 8:97-108). The hematopoietic cell subsets may be flow sorted on the basis of their forward and orthogonal light scatter characteristics (Paffett-Lugassy et al. (2005) Methods Mol. Med., 105:171-98). Cytospins may be prepared on slides from aliquots of the sorted cells and the slides may be stained with Giemsa and May-Grunwald stains to visualize the blood cell lineages and verify separation. The sorted cells may be utilized for qRT-PCR analysis using the same methods as described above for the temporal characterization of zebrafish mll expression in unsorted blood cell populations. In addition, in situ hybridization may be performed on cytospins of the sorted blood cell populations with the same digoxigenin labeled zebrafish mll antisense riboprobe used for WISH on the whole-mounted fish above.

Another strategy which may be employed involves double WISH. Since both primitive and definitive hematopoiesis in zebrafish are characterized by well described spatial and temporal patterns of hematopoietic transcription factor gene expression (Hsu et al. (2001) Curr. Opin. Hematol., 8:245-51; Song et al. (2004) PNAS 101:16240-5; Onnebo et al. (2005) Exp. Hematol., 33:182-8; Hsia et al. (2005) Exp. Hematol., 33:1007-14; Amatruda et al. (1999) Dev. Biol., 216:1-15), double WISH may be performed combining probes for specific blood cell genes with the zebrafish mll probe. For example, scl/tal-1 is expressed at 10 hpf in the PLM indicating initiation of HSC formation, pu.1(sp1) is expressed in the ALM at 12 hpf, signaling commitment to myeloid lineage, c-myb is expressed at 18 hpf in erythroid cells in the ICM. Other blood cell genes that can be interrogated by double WISH with zebrafish mll are the stem cell gene lmo2, gata1 and αglobin associated with erythroid differentiation, runx1, cebpα, l-plastin and mpo associated with myeloid differentiation, as well as the rag1 lymphoid marker. In these experiments, a given fluorescein labeled antisense probe to a blood cell gene of interest may first be used in separate WISH analyses to create a frame of reference (Paffett-Lugassy et al. (2005) Methods Mol. Med., 105:171-98). The same fluorescein labeled antisense probe for the blood cell gene of interest may be used in a simultaneous hybridization with the digoxigenin labeled antisense zebrafish mll probe, followed by detection of the probes with appropriate alkaline phosphatase-conjugated antibodies. The co-expression of zebrafish mll with specific blood cell genes during the development of the zebrafish embryo and in the zebrafish adult may form a foundation for additional experiments on the role of zebrafish mll in hematopoietic cell differentiation.

Temporal RNase protection assays may also be used to detect whether zebrafish mll transcripts in wild type zebrafish can be scrambled or otherwise differ in a developmental manner. A long recognized but little understood finding in the MLL field is that of exon scrambling (Megonigal et al. (2000) PNAS 97:9597-602; Caldas et al. (1998) Gene 208:167-76). Exon scrambling of MLL RNA occurs when exons are joined in a different order than in the genomic sequence but, more often than not, using accurate splice junctions. Scrambled transcripts can be generated from both normal and translocated MLL alleles and detected in both normal and leukemic cells. Zebrafish provide a unique developmental model to investigate MLL exon scrambling. RNAse protection assays may detect alternative splicing or sequence polymorphisms as well as exon scrambling. To perform these assays, [α³²P] dCTP labeled riboprobes may be reverse transcribed from the zebrafish mll cDNA-containing plasmid and hybridized to total RNAs from whole wild-type zebrafish embryos and adults, followed by RNAse T1 digestion (Chatterjee et al. (2005) Dev. Dyn., 233:890-906; Felix et al. (1992) J. Clin. Invest., 89:640-7). Detection of any smaller fragments may indicate incomplete protection of the full-length probe due to sequence differences. Riboprobes to smaller transcript regions may also be generated to localize any differences, which may be studied further by RT-PCR and sequencing of the products. If zebrafish mll exon scrambling or alternative splicing is detected, then tissue specific expression of the variant transcripts may be examined further by temporal RNase protection assays of hematopoietic cells collected from the heart, as well as other hematopoietic and non-hematopoietic tissues. The detection of scrambled transcripts or alternatively spliced transcripts with temporal-specific or tissue-specific patterns of expression would suggest that transcript variation has a developmental function. The corresponding full-length cDNAs may be cloned and sequenced and used in functional studies if temporal-specific or tissue-specific exon scrambling or alternative splicing is detected.

As shown herein, zebrafish mll^{MOE2I2} knockdown embryos exhibited a profound developmental and hematopoietic phenotype that links the MLL gene product to broad molecular cellular pathways. The observed phenotype is a consequence of interplay of mll in pathways that control apoptosis, differentiation, angiogenesis and cell proliferation. Mll expression may be altered in order to investigate the broader molecular cellular pathways in which mll may have a function. For example, one change in expression is zebrafish mll depletion. This may be accomplished using morpholino antisense oligonucleotides, as described hereinbelow. Morpholino antisense oligonucleotides are synthetic DNA analogs that can inhibit translation by targeting the 5′ UTR (Heasman, J. (2002) Dev. Biol., 243:209-14) or block proper splicing of pre-mRNA by targeting splice junctions (Draper et al. (2001) Genesis 30:154-6). The mllMOE2I2 resulted in a profound embryonic phenotype. Additional morpholinos against the same mRNA may be utilized to ensure that the same phenotype is generated. For example, a splice acceptor site morpholino (mllMOI4E5) may be utilized as well as a morpholino based on the 5′ UTR sequence. A gradation of morpholino doses may also be tested to determine whether different amounts of morpholino knockdown are associated with gradations in the phenotype. As another test of specificity of the phenotype, mRNA may be transcribed in vitro from the zebrafish mll cDNA and the zebrafish mll mRNA and morpholino constructs may be co-injected into the same embryos to determine if the morphant phenotype can be rescued.

Mll mutant embryos may also be generated by retroviral insertional mutagenesis to further characterize the effect of mll depletion in a true genetic mutant. Zebrafish lines with mll disruption by retroviral insertional mutagenesis by injection of a 7 kb retrovirus into embryos may be studied as a second avenue to understanding the consequences of zebrafish mll depletion. Znomics, Inc. (Portland, Oreg.) has available several lines with retroviral insertion sites in introns or exons of the zebrafish mll gene. The morphology of the animals may be examined microscopically to identify a mutant that phenocopies the abnormalities in the embryos after morpholino knockdown to be used for further studies. Heterozygote embryos may be grown to adults and bred to generate heterozygous and homozygous mutants. Fish in which mll has been disrupted by retroviral insertional mutagenesis may be used to further characterize the effects of mll disruption on the development of the embryo in general and on the hematopoietic system throughout the lifespan of the animal.

The function of zebrafish mll during embryonic hematopoiesis may also be elucidated by examining the effect of overexpressing zebrafish mll mRNA in wild-type zebrafish embryos. The embryos may be injected with an expression vector comprising zebrafish mll or with in vitro transcribed zebrafish mll RNA over a range of concentrations (e.g. 7, 15, 30 μg) (Davidson et al. (2003) Nature 425:300-6) and the effects of zebrafish mll overexpression on embryonic development and hematopoiesis may be studied.

The spatio-temporal patterns of zebrafish mll mRNA expression in the whole animal, specific blood compartments and specific tissues may be studied with each manipulation that either decreases or increases zebrafish mll expression, and comparisons may be made to the unmanipulated embryos and fish at the same stage of development.

Several lines of evidence suggest that there are either direct or indirect interactions of MLL with apoptosis regulation. First, leukemias in patients with MLL translocations have imbalanced expression of BCL-2 mRNA, which encodes the cardinal anti-apoptotic regulator in the intrinsic cell death pathway. Next, murine Mll^{-/-} embryos exhibit increased apoptosis as evidenced by increased TUNEL staining of the hypoplastic branchial arches. As stated hereinbelow, observations of neuronal and hematopoietic defects suggest that mllMOE2I2 zebrafish phenocopy features of this murine knockout. These observations indicate that MLL alterations disrupt the homeostatic balance of cell death and cell survival factors (Reed, J C (2003) Cancer Cell 3:17-22) that determine apoptosis. The recent cloning of functional homologues of mammalian BCL-2 multi-domain and BH3 only family proteins in zebrafish by Kratz et al. indicates that there is a high degree of evolutionary conservation between zebrafish and mammals (Kratz et al. (2006) Cell Death Differ., 13:1631-40). A series of temporal compound WISH experiments may be performed in order to overlay the developmental-specific expression of normal zebrafish mll mRNA with the developmental-specific expression of zbcl2 family members and decipher with which bcl2 family members normal zebrafish mll is most likely to have interactions. Then compound WISH experiments on zebrafish mll and each relevant zbcl2 family member may be performed on embryos in which mll expression has been altered by morpholino knockdown, retroviral insertional mutagenesis, or mRNA overexpression in order to determine how expression of zbcl2 family members may be altered by altering zebrafish mll expression. Kratz et al. also have determined that expression patterns of particular zbcl2 family members in adult zebrafish exhibit tissue specificity. Tissue specific expression patterns of zbcl2 family members may be studied by compound WISH in wild-type adult zebrafish and heterozygous and homozygous adult zebrafish mll retroviral mutants and quantified in dissected tissues from these fish using RT-PCR.

The compound WISH experiments on embryos in which zebrafish mll has been depleted will likely reveal increased expression of the pro-apoptotic family members and/or decreased expression of anti-apoptotic family members. Interesting, Kratz et al. observed that ectopic expression of certain pro-apoptotic bcl2 family members (zbax1, zbax2, zbok1 and zbok2) caused increased apoptosis manifesting as blastomere and yolk cell disintegration, and that apoptosis induced by pro-death family members zbid, zbmf1, zbmf2, zpuma, znoxa and zbax could be rescued with by expression of anti-apoptotic family members zbip1, zmcl-1a and mcl-1b. In order to further investigate the proposed interaction between zebrafish mll and pro-apoptotic family members, a compound morpholino knockdown experiment may be performed to determine if depletion of the relevant pro-apoptotic family member can rescue the morphant phenotype of zebrafish mll depletion. Rescue of any aspects of the phenotype of zebrafish mll depletion by knocking down expression of the pro-apoptotic mRNA may provide further evidence that zebrafish mll is involved in apoptosis regulation. Any suggestion of potential interactions between zebrafish mll and the pro-death zbcl-2 family members may also be investigated further by determining if the embryonic phenotype from overexpression of in vitro transcribed mRNA for the relevant pro-death zbcl-2 family can be rescued by simultaneous overexpression of zebrafish mll.

Conversely, increased zebrafish mll expression may be associated with decreased pro-apoptotic gene expression and/or increased anti-apoptotic gene expression. Therefore, in the compound WISH experiments embryos in which zebrafish mll mRNA is overexpressed may be used in order to determine if increased mll expression is associated decreased expression of any of the pro-apoptotic bcl-2 family members or increased expression of any of the anti-apoptotic family members. Another observation made by Kratz et al. is that the anti-apoptotic zmcl-1a and zmcl-1b family members are critical to normal embryonic development and that zebrafish in which these genes are depleted have decreased survival. Furthermore, it has been established that Mcl-1 is critical to maintaining hematopoietic stem cells and progenitor cells in a murine model (Opferman et al. Science 307:1101-4). Therefore, the next question that will be addressed is whether zebrafish mll has selective interactions with the zmcl-1a and zmcl-1b anti-apoptotic zbcl2 family members. If the compound WISH experiments reveal that zebrafish mll overexpression is associated with increased expression of zmcl-1a and zmcl-1b or other anti-apoptotic zbcl2 family members or, conversely, that mll depletion is associated with decreased expression of anti-apoptotic family members, then additional experiments may be performed in order to determine if the phenotype of depletion of the relevant anti-apoptotic zbcl2 family member mRNA from morpholino knockdown can be rescued by co-injection with zebrafish mll mRNA. Further experiments may also be designed to determine whether overexpression of the anti-apoptotic zbcl2 family member mRNA is able rescue phenotype of mll depletion.

Zebrafish mll depletion may be associated with increased apoptosis and, conversely, that zebrafish mll overexpression is associated with decreased apoptosis as a consequence of various interactions with particular zbcl-2 family members. If a specific interaction is discovered between zebrafish mll and a specific pro- or anti-apoptotic family member, then blood cells from zebrafish with the relevant alterations in zebrafish mll expression and expression of zbcl2 family members may be collected via cardiac puncture and flow sorted for more detailed temporal analyses in order to characterize the interaction further in specific blood cell populations. In addition to the characterizing the relationship between temporal and spatial expression patterns of zebrafish mll and those of zbcl2 family members in zebrafish embryos with altered zebrafish mll expression as well as in homozygous and heterozygous mll retroviral mutant zebrafish adult fish, several complementary strategies to detect whether manipulating zebrafish mll expression alone may be employed and combined with the various manipulations of zbcl2 family member mRNAs has effects on apoptosis. Strategies to detect and quantify apoptosis that have been used in zebrafish include TUNEL staining of whole mounted animals and whole-mount immunohistochemistry with antibody detection of active caspase 3. Additional information may also be gained by through use of the same markers for flow cytometric assays of blood cell populations.

Several lines of evidence support a role of MLL in hematopoietic differentiation. In vitro culture of Mll deficient and haplo-insufficient yolk sac progenitor cells from the murine model demonstrated that myeloid and macrophage differentiation is Mll dependent. Constructs comprising MLL AT hook motifs were shown to promote p21 and p27 upregulation, cell cycle arrest and monocyte maturation. Other evidence that MLL has a role in blood cell development derives from the aberrant expression of B-lymphoid and myeloid surface antigens on leukemia cells in which MLL is altered. In addition, the zebrafish mllMOE21E knockdown embryos exhibited a profound hematopoietic phenotype which supports a role of MLL in blood cell development.

Changes in the composition of blood cell populations caused by decreasing or increasing zebrafish mll expression may be used to further study the role of MLL in hematopoietic differentiation. Zebrafish leukocytes may be studied by flow cytometry. Additionally, antibodies which recognize different B cell populations may be employed. For example, antibodies against zebrafish IgM and IgZ that may be generated to characterize changes in B cell populations from zebrafish mll manipulations. These antibodies may also be used to determine the potential molecular mechanism through which zebrafish mll may control differentiation of the B cell lineage.

Gene expression profiling recently has shown that B lymphoid leukemias with MLL translocations have increased expression of the paired domain transcription factor gene PAX5, which encodes the B-cell lineage specific activator protein (BSAP) (Kohlmann et al. (2005) Leukemia 19:953-64). Not only do the human leukemias overexpress this gene, but also murine models have resulted in leukemias with co-expression of lymphoid and myeloid marker that express Pax5 (Zeisig et al. (2003) Oncogene 22:1629-37). It has also been suggested that PAX5 is involved in the control of B lineage commitment and the suppression of other lineage choices (Urbanek et al. (1994) Cell 79:901-12). Other studies have suggested that the role of PAX5 in controlling commitment to the B cell lineage involves repressing the expression of FLT3 (Holmes et al. (2006) Genes Dev., 20:933-8), a receptor tyrosine kinase that is often activated in leukemias with MLL translocations (Brown et al. (2005) Blood 105:812-20; Brown et al. (2004) Blood). In addition, the silencing of Pax5 in a murine B cell lymphoma model resulted in differentiation along the macrophage lineage (Hodawadekar et al. (2007) Exp. Cell Res., 313:331-40). The nature of this gene makes it an attractive candidate for further evaluation in compound WISH analyses with analyses of zebrafish mll expression using wild type zebrafish and zebrafish in which zebrafish mll expression has been altered. In addition, the zebrafish ortholog of pax5 has been characterized (Pfeffer et al. (1998) Development 125:3063-74) such that interactions of zebrafish mll and zpax5 may be further queried in compound morpholino gene depletion studies and overexpression studies using the anti-IgM and anti-IgZ antibodies to determine how these manipulations result changes in the blood lineages. Notably, the zebrafish mllMOE2IE mutant showed a hindbrain malformation and zpax5 is involved in the development of this area of the brain (Pfeffer et al. (2000) Development 127:1017-28). In addition, the Drosophila homologue of this gene sparkling controls eye development (Fu et al. (1997) Genes Dev., 11:2066-78) and the morphant generated herein also has small eyes.

MLL is proteolytically cleaved into two separate amino and carboxyl terminal proteins. MLL proteolytic cleavage is essential for cell cycle progression. As described herein, the taspase1 site in the zebrafish mll ortholog is evolutionarily conserved and, moreover, that the zebrafish mllE2I2 morphant embryos were characterized by small size, the hallmark feature of the Taspase1 −/− mouse. The proteolytic cleavage of MLL may be developmentally controlled, and cleavage of the translated protein may be spatially and temporally regulated. The zebrafish mll cDNA may be genetically tagged (Giepmans et al. (2006) Science 312:217-24) with different fluorophors at the 5′ and 3′ ends before in vitro transcription of the mRNA and overexpression of the mRNA via micro-injection in the zebrafish embryos. This allows the exploitation of the transparent nature of the zebrafish embryos to visualize, follow and locate within the live embryos the dynamics of both ends of the marked protein in a temporal and spatial fashion. There is an increasing published experience that the choice of fluorescent proteins can be optimized for brightness and expression (Shaner et al. (2005) Nat. Methods 2:905-9). The literature suggests that a combination of mCherry and the newer variant of EGFP, Emerald, would be reasonable choices for the labeling. The expression patterns of the cleaved and non-cleaved zebrafish mll fluorescent protein as visualized microscopically may be correlated with the expression of the taspase1 gene and cell cycle control genes that have been shown to be regulated by the cleavage state of the murine Mll oncoprotein including E2Fs, and cyclins E (ccne), A (ccna2), and B (ccna2) and p16Ink4A. In addition, if there are developmentally regulated variants of zebrafish mll transcripts due to exon scrambling or alternative splicing, the cloned zebrafish mll variants may also be genetically dual labeled in order to determine if variation at the transcript level is a way to regulate the cleavage of the eventual gene product. The ability to visualize the dynamic distribution of the separate amino and carboxyl fragments of the protein with transrepression and transactivation properties also may suggest that they have separate functions apart from those directed by to the single macromolecular protein complex in which they reassociate if they are found in different locations in the embryos. This would be of interest because constructs comprising MLL AT hook motifs have previously been shown to promote p21 and p27 upregulation, cell cycle arrest and monocyte differentiation.

A striking feature of the zebrafish mllMOE2I2 morphant embryos was the neuronal defect that appears to phenocopy the hindbrain abnormality in zebrafish following runx1 depletion. Another characteristic in zebrafish following runx1 depletion in addition to the hematopoietic and neuronal defects was incomplete vasculature formation (Kalev-Zylinska et al. (2002) Development 129:2015-30). The zebrafish mllMOE2I2 morphant embryos also showed less blood in the heart and at the ventral surface compared to the wild type controls. Defective angiogenesis has also been characterized extensively in Aml1 (Runx1) null mice and, in this model, there was defective angiogenesis in the head and pericardium (Takakura et al. (2000) Cell 102:199-209). The similarities to the zebrafish mllMOE2I2 morphant suggest MLL may also have a role in vasculature formation. Curiously, the defective angiogenesis in the Aml1 mutant mice was rescued not only by HSCs but also by angiopoietin-1 (Ang1), which is expressed in HSCs. WISH analysis of zebrafish embryos with zebrafish mll depletion or forced overexpression may be used to examine mRNA expression of the vasculature markers flk-1 and Ang1 and compound WISH analysis to overlay the expression of these markers with zebrafish mll. A search of the bioinformatics databases reveals that zebrafish counterpart of Ang1 is angpt1. Additionally, the homozygous and heterozygous zebrafish mll retroviral insertional mutant embryos and fish will be informative as to whether there are gradations in the defective vasculature phenotype. The other question raised by the overlapping phenotype is whether zrunx1 and zebrafish mll are involved in the control of overlapping pathways in the cell. To examine this possibility further, it may be determined if forced overexpression of zebrafish mll mRNA can rescue any aspects of the morphant phenotype associated with runx1 depletion as well as whether flk-1 or angpt1 depletion by morpholino knockdown phenocopy any aspects of the embryos with zebrafish mll depletion.

The role of MLL translocations with specific partner genes may be studied in transgenic zebrafish. For example, the MLL-GAS7 fusion protein generated by a recurrent translocation in human AML may be studied. MLL-FRYL, the indolent phenotype of which in patients is at the opposite end of the clinical spectrum to MLL-GAS7, is another tranlocation that may be studied.

Transgenic technology to overexpress a gene of interest through the use of tissue-restricted gene promoters in zebrafish has been well described (Langenau et al. (2005) Blood 105:3278-85). The transgenic embryo carrying a tissue-specific promoter linked to a GFP reporter gene can provide a rapid, real time in vivo system for analyzing spatial and temporal expression of the transgene and its phenotypic consequences (Chalfie et al. (1994) Science 263:802-5). To directly assess if zebrafish are a useful model for the study of myeloid MLL-related leukemogenesis, a full-length human 5′-MLL-GAS7-3′ cDNA based on that utilized in the murine retroviral transplantation model may be generated. This full-length cDNA may be cloned into the EGFP-C1 vector expression vector, utilizing zebrafish spi1 promoter regulatory elements for targeted expression of the transgene. The promoter of the pu.1 (spi1) early myeloid development transcription factor was selected to target the expression of the transgene to early myeloid precursors that best simulate the affected cells in the clinical AML. The spi1-EGFP-MLL-GAS7 construct may be injected at the single-cell stage of development generating F0 founder fish mosaic for expression of the transgene. To confirm appropriate expression of the spi1-EGFP-MLL-GAS7 transgene, the embryos may be analyzed under a fluorescent microscope for GFP expression. The presence of human MLL-GAS7 protein may be analyzed by Western blot with an MLL-specific antibody. Embryos injected with vector alone or with the spi1-EGFP reporter construct may be used as negative controls.

To determine the effects of MLL-GAS7, GFP fluorescence in the embryos may be serially monitored microscopically and compared to the controls with particular attention to perturbations in the distribution of the signal in the hematopoietic compartments of the fish. The cytology of blood smears collected from the heart after 24 hpf when circulation becomes visible may be stained with to characterize the morphology of the cells. To gain further insight into the perturbed cell population the embryos injected with spi1-EGFP-GAS7 as well as the controls will be examined for expression of HSC and blood lineage transcription factor genes by WISH analysis of the whole mounted embryos exactly as described in the aims above. Anti-IgM and anti-IgZ antibodies in combination with the light-scatter characteristics of blood leukocytes will enable flow cytometric evaluation and quantification of the changes in leukocyte composition caused by spi1-EGFP-MLL-GAS7 as well as better sorting for more detailed analyses of the effects of the transgene on specific blood cell populations. The cells may also be examined for expression of IgM+, which has been found in B cells with phagocytic properties in the rainbow trout to examine the mixed lineage nature of the leukemia in an evolutionary context. Stable inducible transgenic zebrafish lines may also be produced.

The zebrafish model provides a new and powerful model system to decipher the role of mll in zebrafish embryogenesis, determine its place in zebrafish blood cell development and in leukemogenesis. Leukemias with MLL translocations are refractory to current treatments. The zebrafish of the instant invention provide a rapid screening tool to test anti-leukemic agents targeting leukemias with MLL translocations.

### VI. Screening Methods

Screening methods for the discovery of compounds which lessen a phenotype associated with the reduced activity of mll are provided. Transgenic animals, particularly transgenic zebrafish, of the instant invention are contacted with at least one test compound. The transgenic animal may have increased or decreased mll expression and/or may express mll linked to a partner gene as in the leukemias described hereinabove. Compounds are tested for their ability to lessen or even eliminate a phenotype (i.e., return to or approach a wild-type phenotype) associated with the altered mll expression (e.g., reduced or eliminated expression of mll). For example, the compound may correct one of the exhibited phenotypes described hereinbelow, such as, hematopoietic defects (e.g., lack of erythroid cells in heart/ventral anterior yolk sac), neuronal defects, small size, smaller eyes, delayed development, aberrant head protrusion, and hindbrain abnormalities.

In one embodiment, the target compound may be optimized by testing chemical variants of a target compound through a combinatorial chemistry approach. The test compounds and chemical variants may also be tested for properties such as, but not limited to, enhanced efficacy, enhanced solubility, and/or toxicity.

General screening methods are also provided in U.S. Patent Application Publication 20050155087, 20050244808, and 20040117867.

Compounds identified by the instant screening methods may be considered anti-cancer compounds, more specifically anti-leukaemia compounds. The identified compounds can also be used to control hematopoiesis.

In another embodiment, the transgenic animals of the instant invention may be screened to identify other phenotypes associated with altered MLL expression, including MLL translocation expression. For example, the effects of the altered levels of MLL on the differentiation, lineage, quantity, and immunophenotype of blood cell types may be determined.

The following examples are provided to illustrate various embodiments of the present invention. They are not intended to limit the invention in any way.

### EXAMPLE I

### Zebrafish MLL Sequence

Bioinformatics tools were used first to determine the existence and relationship of a zebrafish MLL ortholog to human MLL. BLASTP searching on the NCBI database server (www.ncbi.nlm.nih.gov/BLAST/) using the full-length human MLL protein (GenBank Accession no. NP_{—}005924) as the reference sequence identified two putative “similar to MLL proteins” containing 2251 amino acids (GenBank no. XP_{—}685032) and 1904 amino acids (GenBank no. XP_{—}685116).

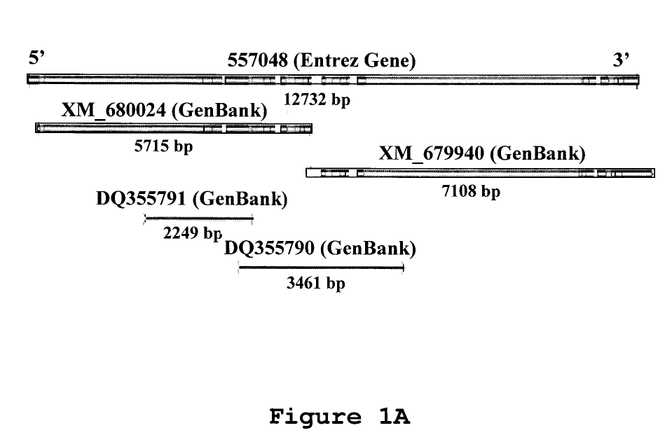

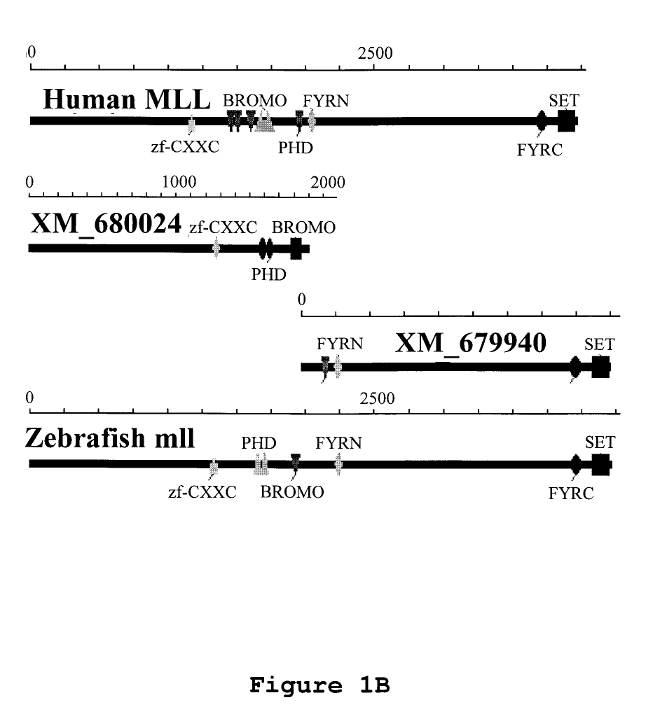

GenBank entries for two predicted transcript sequences, XM_{—}680024 and XM_{—}679940, which corresponded to the two “similar to MLL proteins,” were also identified using BLAST. The two predicted transcript sequences are in close proximity to each other and span positions 31,979,440 to 31,961,790 and 31,961,430 to 31,952,674, respectively, on zebrafish chromosome 15. The more 5′ 5715-base sequence XM_{—}680024 contained 17 predicted exons, whereas there were 7108 bases and 18 predicted exons in XM_{—}679940. Furthermore, ENSEMBL (www.ensembl.org) projected that the two sequences comprised a single larger transcript comprising 35 exons and 12732 bases (Entrez Gene 557048). Most recently, Sun et al. deposited in the GenBank database partial transcript sequences at the central portion of this region cloned from zebrafish kidney marrow (DQ355790 and DQ355791). The relationship of the two predicted “similar to MLL” transcript sequences to the predicted single transcript (Entrez Gene 557048) and the two central partial sequences is shown in FIG. 1A.

The GNOMON gene prediction tool, which evaluates transcripts and proteins aligned to a genome (www.ncbi.nlm.nih.gov/genome/), was used to predict the genomic structure(s) corresponding to the two zebrafish “similar to MLL” protein sequences (GenBank nos. XP_{—}685032 and XP_{—}685116) in zebrafish genomic DNA. The results of GNOMON analysis also predicted that a single genomic sequence (GenBank accession no. NW_{—}633640) matched both protein sequences.

Next, CDART analysis tools (www.ncbi.nlm.nih.gov/Structure/lexington/lexington.cgi) were employed in order to compare human MLL and the two zebrafish “similar to MLL” proteins. The CDART algorithm finds protein similarities across significant evolutionary distances using protein domain architecture, i.e. the sequential order of conserved domains in proteins, rather than direct sequence similarity (Geer et al. (2002) Genome Res., 12:1619-23). Interestingly, this analysis suggested that the two predicted proteins together resembled mammalian MLL in its entirety and that important domains of human and mouse MLL including the CXXC domain, bromodomain, PHD zinc fingers, FYRN, FYRC and SET domain all were present in a hypothetical single zebrafish protein (FIG. 1B).

While there can be one-to-many and many-to-many relationships (Tatusov et al. (1997) Science, 278:631-7) between human and zebrafish genes due to gene duplication over evolutionary distance, the predictions of a single gene (GenBank accession no. NW_{—}633640) and single larger transcript (Entrez Gene 557048) matching human MLL is most consistent with a one-to-one relationship. Another question in comparing the predicted zebrafish mll gene to human MLL was whether zebrafish mll is an ortholog, i.e. a gene evolved from a common ancestral gene with the same function, or a paralog that arose by duplication with a different function (Tatusov et al. (1997) Science, 278:631-7). Because the syntenic relationship (Barbazuk et al. (2000) Genome Res., 10:1351-8) between genes is an important predictor of functional similarity, the Ensembl database was employed to examine synteny between the predicted zebrafish mll and human MLL genes. Ensembl genes were compared within 1.6 Mb regions centered around human MLL at chromosome band 11q23 and putative zebrafish mll ortholog on chromosome 15. This analysis revealed that there was a conserved block of synteny surrounding zebrafish mll and human MLL containing several linked genes. In addition, zebrafish mll and human MLL are in the same map order in similar uninterrupted segments with the gene for ubiquitination factor E4A (human (UBE4A) and zebrafish (557121) genes). Other genes found in same map order in similar uninterrupted segments include human NLRX1, PDZD3, HMBS, CBL, and ABCG4 and zebrafish 557335, 557269, zgc:110690, zgc:92560, and ENSDART00000089166, respectively.

Thus the existence of a single zebrafish mll gene with functional similarity to human MLL was supported by several gene and protein prediction methods as well as the syntenic relationship indicated by the respective surrounding genes. This prediction was further strengthened by the prior characterization in pufferfish (fugu), a teleost more closely related to the zebrafish, of a single MLL-like gene with structural similarity and high overall sequence identity to human MLL (Caldas et al. (1998) Oncogene, 16:3233-41).

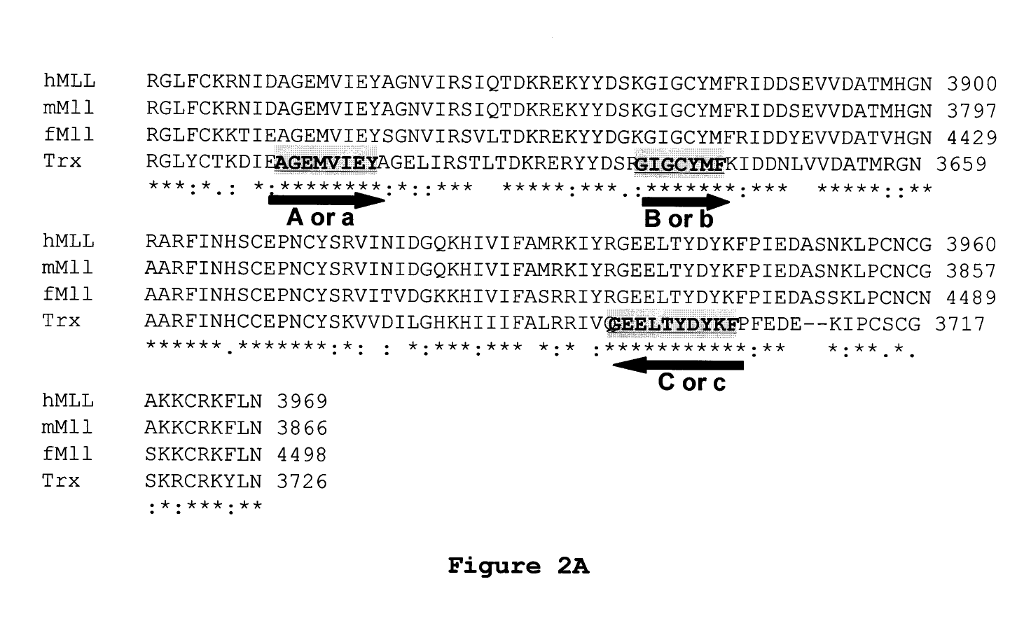

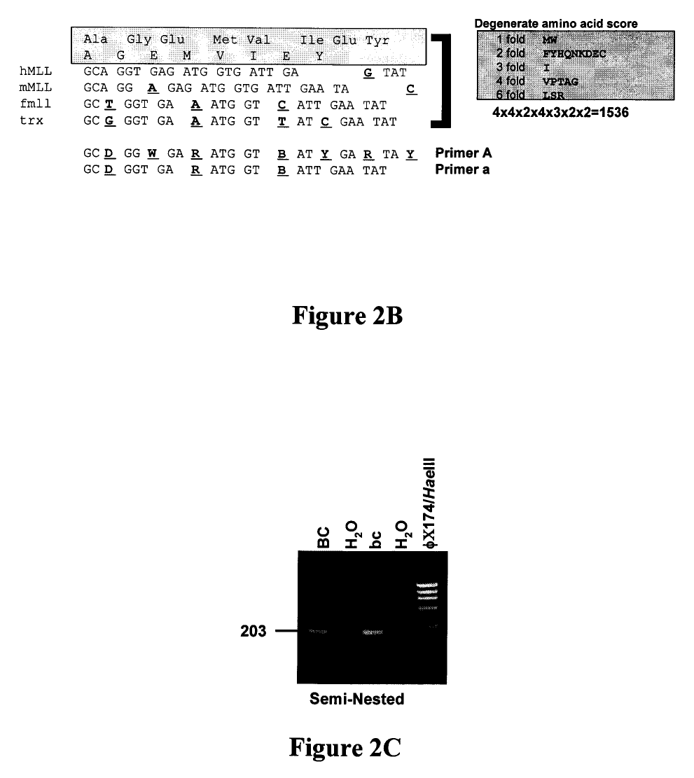

The above experiments determined whether cross-species counterparts of amino acid sequences of highly conserved domains of MLL could be used to identify the corresponding orthologous zebrafish transcript sequence. The amino acid sequences from MLL domains determined by ClustalW analysis (www.ebi.ac.uk/clustalw/) to be the most highly conserved (namely the SET domains) across species from human through mouse, pufferfish and fly, were used to design degenerate primers. Degenerate primers were designed from a region of low degeneracy. Fold degeneracy of amino acid sequences in human, mouse, pufferfish (fugu) and Drosophila trx was determined from the product of the degenerate amino acid score (boneslab.bio.ntnu.no/degpcrshortguide.htm) after examining corresponding transcript regions for codons with a mismatched base. Two primers were designed for each highlighted amino acid region in FIG. 2 using the mixed base code (R=A, G; Y=C, T; M=A, C; K=G, T; S=C, G; W=A, T; H=A, C, T; B=C, G, T; V=A, C, G; D=A, G, T; N=A, C, G, T). One (capitalized) counted as degenerate at all positions with >1 mismatch(es) between the 4 species and the second (lower case) incorporated into the primer sequence any consensus base that matched in 3 or 4 of the species. Degenerate primer mixtures A and C or a and c were used in initial PCRs. A 2 μl aliquot of respective initial PCRs was used for semi-nested PCR with degenerate primer mixtures B and C (first lane) or b and c (third lane) (FIG. 2C). RT-PCR produced a 203 bp product. Sequencing showed that products of both semi-nested PCRs corresponded to XM_{—}679940 sequence (99% identity) predicted to be zebrafish ortholog of human MLL. Similarly, products could be generated in an additional degenerate RT-PCR experiment interrogating the transcript region corresponding to the PHD.

These studies using degenerate primers demonstrate that transcript regions encoding specific MLL functional domains are highly conserved throughout evolution. Not only is there high cross-species homology at the amino acid sequence level (FIG. 2), but also the cross-species counterparts of amino acid sequences could be used to generate the predicted transcript, providing the first experimental evidence that the transcript represented the bona fide orthologous mll gene from the zebrafish species.

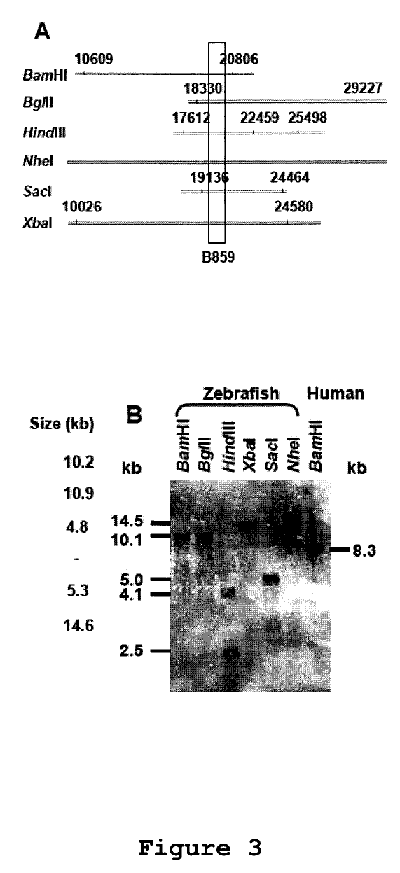

Similarly, cross-species Southern blot analysis of zebrafish genomic DNA was performed using the B859 fragment (Gu et al. (1992) Cell, 71:701-8) containing exons 5-11 of the human ALL-1 (MLL) cDNA to determine if the human probe would detect the predicted zebrafish mll gene. First, restriction maps were simulated for the enzymes BamHI, BglII, HindIII, NheI, SacI and XbaI from a projected 36,662 bp genomic sequence corresponding to the predicted single zebrafish mll cDNA (Entrez Gene 557048), and the region of highest homology to the human probe was used to project the restriction fragment sizes that would be detected (FIG. 3A). Approximately 90 bases of the predicted zebrafish mll cDNA sequence match the probe exactly.

FIG. 3B provides an autoradiograph of zebrafish genomic DNAs and normal human subject peripheral blood lymphocyte DNA after probing with B859. DNA was extracted from a whole wild type adult zebrafish using DNeasy tissue kit (Qiagen, Valencia, Calif.). 20 μg of zebrafish DNA was digested to completion with the indicated enzyme. 10 μg of BamHI-digested human DNA was included as a positive control. Conditions for electrophoresis, Southern transfer, nick translation and hybridization were those employed routinely for human DNAs (Felix et al. (1997) Blood, 90:4679-86). The sizes and numbers of hybridizing fragments in zebrafish genomic DNA exactly matched those predicted, except with HindIII where a single fragment was expected and two fragments were detected (FIG. 3B). This difference is likely due to generation of the zebrafish mll genomic sequence with a gene prediction tool. Therefore, in this experiment the genomic region corresponding to the human MLL bcr was simulated and detected in zebrafish mll.

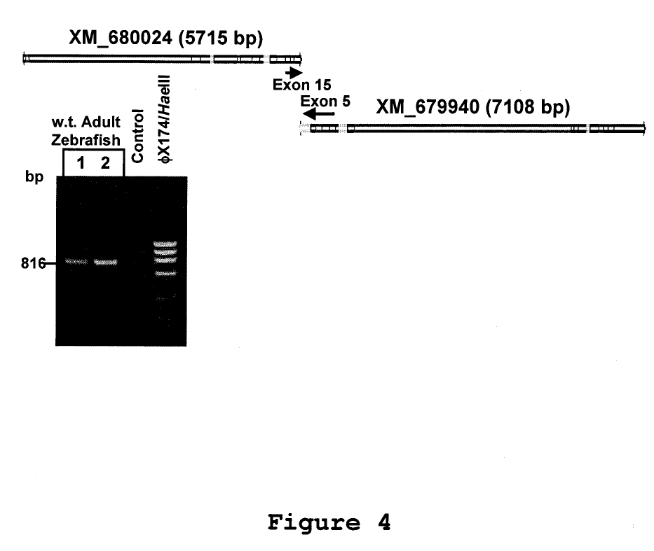

Additional experiments utilized reverse transcriptase PCR(RT-PCR) analysis of total RNA from a whole wild-type adult zebrafish in order to investigate whether the two predicted “similar to MLL proteins”, which, in turn, predicted transcript sequences in close proximity to each other on chromosome 15, were derived from a single gene encoding a putative zebrafish mll with functional domains similar to human MLL. Total RNA was extracted from a whole wild-type adult zebrafish using TRIZOL reagent (Invitrogen; Carlsbad, Calif.). Oligo(dT) primed first strand cDNA was synthesized from 5 μg of total RNA using SuperScript™ II reverse transcriptase (Invitrogen). Sense primer 5′-GAGAGCAGGAAAGCCAACAG-3′ (SEQ ID NO: 16) from exon 15 of XM_{—}680024 and antisense primer 5′-TGGTTCAAGTCCATTAACAAATTTTCT-3′ (SEQ ID NO: 17) from exon 5 of XM_{—}679940 generated a single product that spanned both cDNAs, sequencing of which indicated that the two cDNAs are partial 5′ and 3′ sequences of a single gene (FIG. 4).

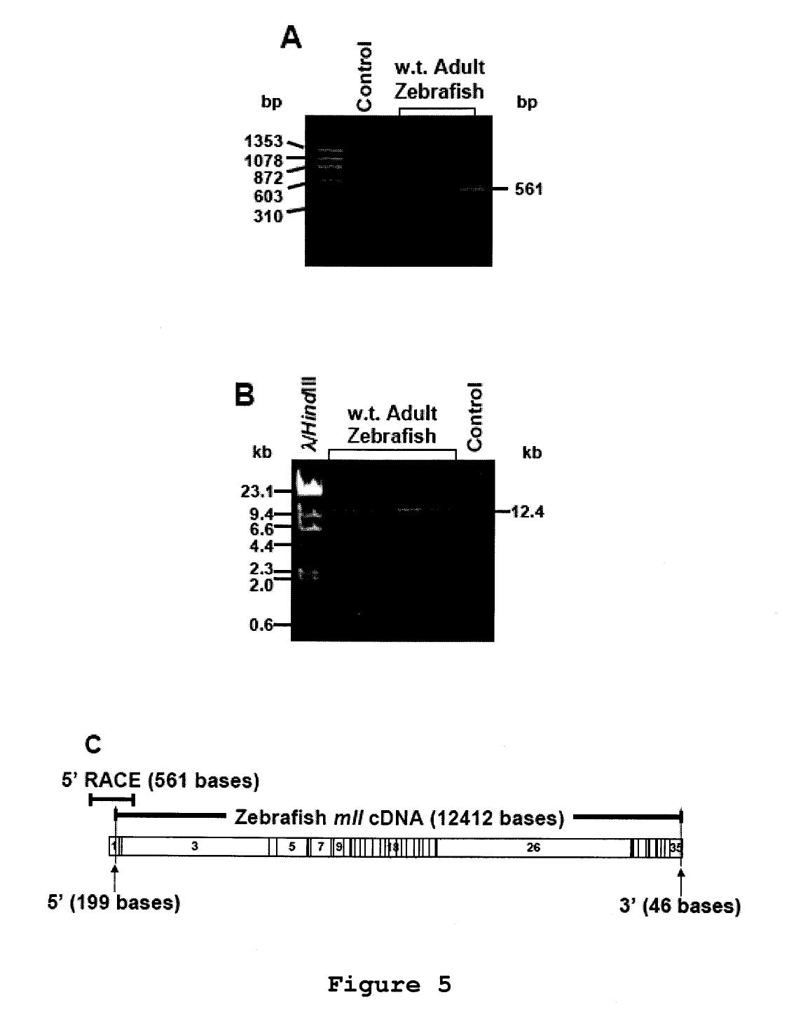

Having determined that the zebrafish mll ortholog to human MLL was a single gene on chromosome 15, the strategies of 5′ Rapid Amplification of cDNA ends (RACE) PCR and long-distance PCR were applied in order to attain and characterize a full length zebrafish mll cDNA. As summarized hereinabove and in FIG. 1, the bioinformatics databases contain only partial sequences of predicted mll cDNAs and ˜5 kb of cloned sequence from the central region of the gene. Moreover, the cDNAs derived with gene prediction tools are not precise representations of the sequence especially at the exon boundaries. The 5′ RACE procedure (Frohman et al. (1988) PNAS, 85:8998-9002) was utilized and information on the predicted sequence of exon 3 in zebrafish mll cDNA to analyze whole wild-type adult zebrafish total RNA and obtained the 561 bp product shown in FIG. 5A containing the unknown 5′ UTR. Specifically, pooled aliquots of total RNAs from two whole wild-type adult zebrafish extracted with TRIZOL reagent (Invitrogen) were used. First strand cDNA was synthesized from 5 μg of total RNA using the SuperScript™ II 5′ RACE System (Invitrogen) and an antisense gene-specific primer (GSP1) designed from exon 3 of clone XM_{—}680024 (5′-TTTGGCTGACAGAAGCAGGAG-3′; SEQ ID NO: 18). A homopolymeric dC tail was added to the 3′-end of the first strand cDNA using TdT and dCTP. The sense strand was synthesized and amplified by PCR from the dC-tailed first-strand cDNA using Taq DNA polymerase, a deoxyinosine-containing anchor primer provided with the system and a nested antisense gene-specific primer (GSP2) from exon 3 (5′-GCAAAGGGGCTGTTTCAGTA-3′; SEQ ID NO: 19). The 5′ RACE PCR was performed in duplicate. Sequencing demonstrated the 5′ UTR of zebrafish mll. Additionally, oligo-dT primed first strand cDNA was synthesized from total RNA from a whole wild-type adult zebrafish using SuperScript™ II First Strand Synthesis reagents (Invitrogen). RT-PCR was performed using Accuprime High Fidelity Taq Polymerase (Invitrogen) with a sense primer from exon 1 (5′-AATTTCGGGATGTTTTGGGGGAGTC-3′; SEQ ID NO: 20) and an antisense primer from exon 35 (5′-AGCTTATTGCCTGGTTCTTCGATGG-3′; SEQ ID NO: 21) designed from the sequence of Entrez Gene 557048. Five of 7 reactions generated the predicted 12.4 kb product (FIG. 5B). PCR products were gel-purified using a TOPO XL kit (Invitrogen) and subcloned into a TOPO XL vector (Invitrogen). Subclones with the desired insert were identified by PCR screen of bacterial mini-cultures with the exon 1 and exon 35 primers used for the original PCR. In addition, RNA was prepared from thirty pooled 24 hpf embryos using a RNeasy (Qiagen, Valencia, Calif.). A mixture of oligo-dT primed and random hexamer primed first strand cDNAs, which were generated using SuperScript™ II First Strand Synthesis kit (Invitrogen), was amplified with the same primers as above. Reaction products were gel-purified, cloned into TOPO XL vector and sequenced in entirety. The sequences of the subclones generated from adult and zebrafish embryo RNAs contain all but the 199 bases at the most 5′ end and 46 bases at the most 3′ end of the full-length sequence. A 12412 sequence contig was generated from sequencing two subclones and directly sequencing of products of 3 independent PCRs derived from the embryos. FIG. 5C provides a summary of 5′ UTR and 35-exon overlapping sequence generated as described above, thereby generating a near complete zebrafish mll cDNA. The 199 bases at the most 5′ end, which are not present in the 12412 base subclone, were subsequently obtained by 5′ RACE.

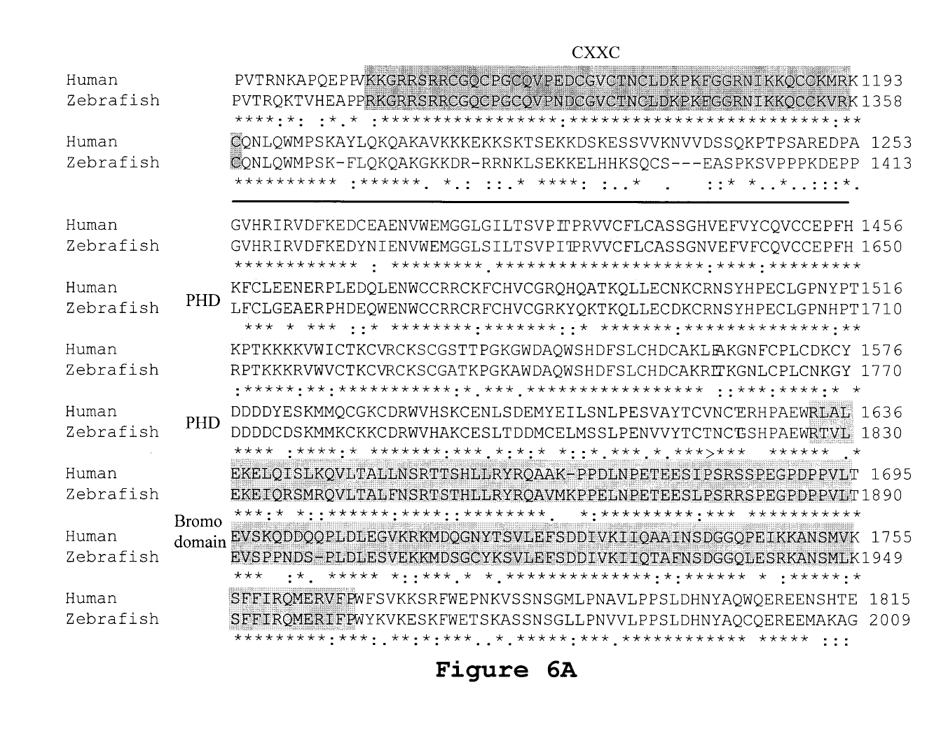

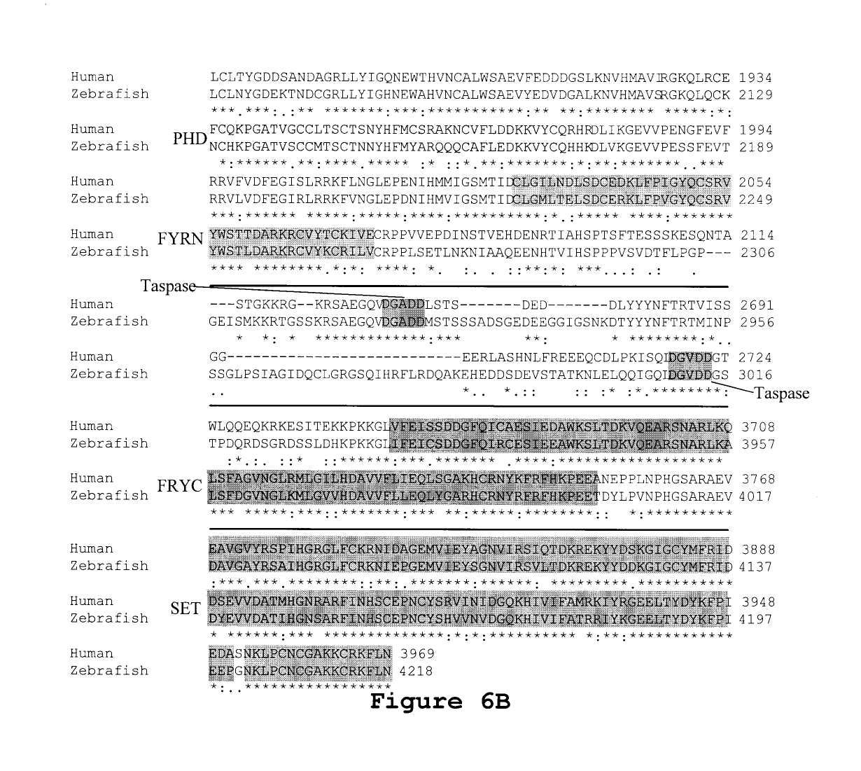

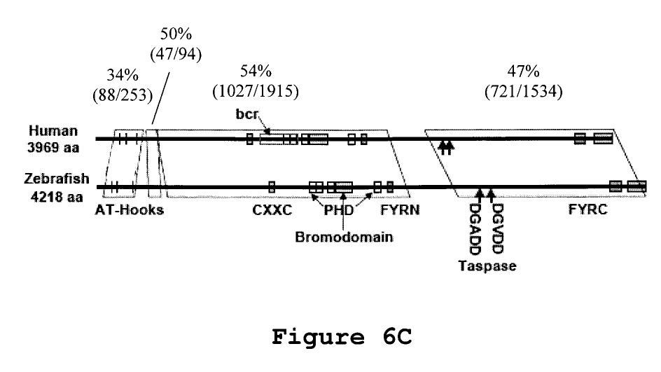

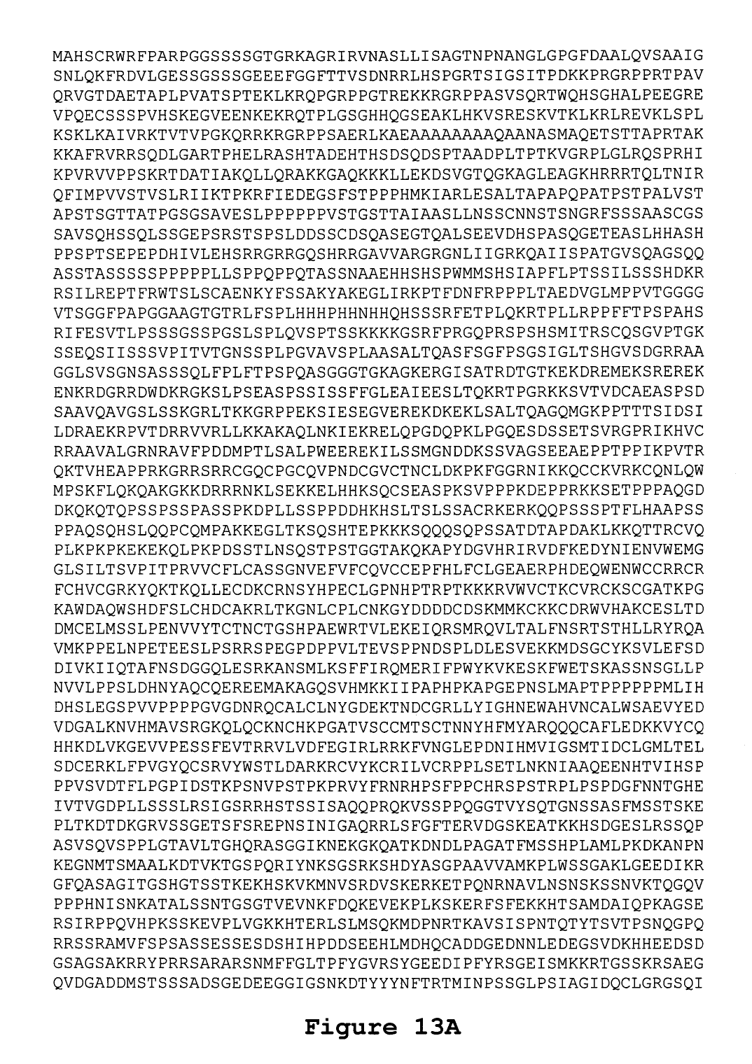

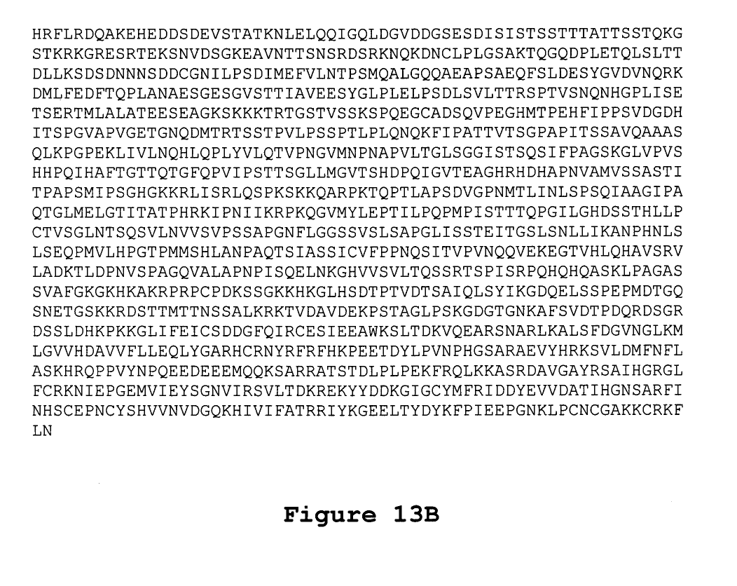

Next, the 12412 bp zebrafish mll cDNA sequence, the 5′ coding sequence, and the 46 missing 3′ bases taken from Entrez Gene 557048 were combined in order to compare the zebrafish mll cDNA and predicted protein to human MLL and its protein product (see FIGS. 12B-12F and 13A-13B). The human MLL cDNA (GenBank Accession no. L04284) contains 11910 bases and 36 exons, while there are 12657 bases and 35 exons in its zebrafish ortholog. ClustalW analysis of the zebrafish mll protein predicted by the cDNA clones that were generated and the 46 missing 3′ bases indicated that zebrafish mll contains all of the same important functional domains as the human protein (GenBank accession no. AAA58669; see FIGS. 6A and 6B). Protein domain alignments were generated using SMART (smart.embl-heidelberg.de/) and NCBI BLAST programs. Human MLL is a 3969 amino acid protein, while there are 4218 amino acids in zebrafish mll with 45.7% sequence identity overall to the human protein. The highest amino acid sequence identity (54%) is in the central portion of the protein containing the plant homology domains, bromodomain and FYRN sequence, but there also is high amino acid sequence identity (50%) in a less well defined amino terminal region, and in the more carboxyl terminal portion of the protein where the taspase cleavage sites, FYRC and SET domain are located.

### EXAMPLE II

### Zebrafish MLL Expression

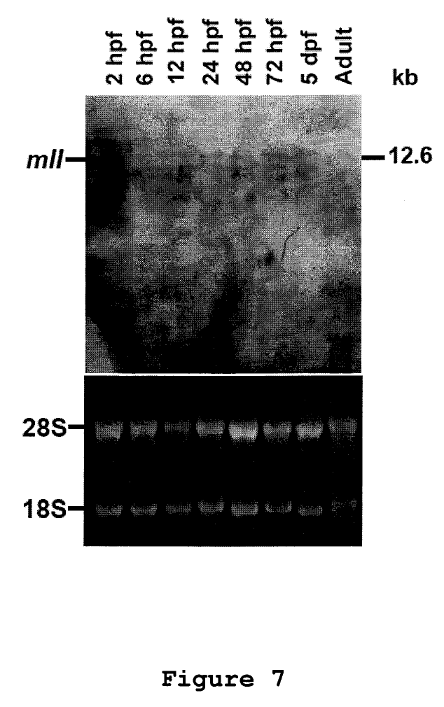

The temporal pattern of mll RNA expression was examined in wild-type zebrafish embryos and whole adults using Northern blot analysis and RT-PCR. Twenty μg of total RNA per lane from whole wild type adult or pooled wild type zebrafish embryos were collected at the indicated times and were probed with the 12.4 kb fragment of zebrafish mll cDNA. The conditions for electrophoresis, transfer, nick translation, and hybridization were those employed for human RNAs (Felix et al. (1987) J. Clin. Invest., 80:545-56) except that no blocking DNA was used in hybridization. As shown in FIG. 7, Northern blot analysis using total RNAs detected an abundant 12.6 kb signal consistent with mll transcript expression in the embryos at 2 hpf, and weak 12.6 kb signals at 24 hpf, 48 hpf, 72 hpf, 5 dpf and in the adult.

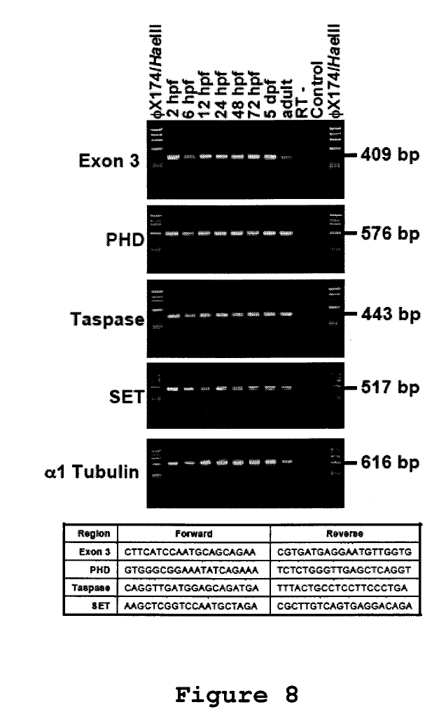

In more sensitive RT-PCR analyses of several different amplicons from exon 3 and regions encoding the PHD, taspase cleavage sites and SET domain were studied. Products were detected at all timepoints and for every amplicon that was tested including as early as 2 hpf, throughout embryonic development, and in the adult sample (FIG. 8). Specifically, RNA was extracted using RNeasy (Qiagen, Valencia, Calif.) from pooled wild-type zebrafish embryos harvested at the indicated times and from a whole wild-type adult zebrafish. One μg of total RNAs were used to synthesize first-strand cDNAs using random hexamers (Applied Biosystems). RT-PCR was performed with High Fidelity Taq polymerase (Roche, Indianapolis, Ind.) and primers corresponding to specified regions of zebrafish mll or to α1 tubulin. Wherever possible (e.g., PHD and SET), primers were designed to generate products that would cross exon junctions. Sequencing confirmed respective zebrafish mll transcript sequences.

It has been previously demonstrated that zygotic gene expression in zebrafish does not begin until 3 hpf and that maternal transcripts are degraded by 5 hpf, after which all transcripts are zygotic (Chatterjee et al. (2005) Dev Dyn, 233:890-906; Christie et al. (2004) Am. J. Physiol. Heart Circ. Physiol., 286:H1623-32). Therefore, the detection of a signal on Northern blot analysis and the generation of RT-PCR products at the earliest timepoint (2 hpf) is consistent with the presence of maternally supplied mll transcripts in the embryo. This finding indicates that maternal mll mRNA is important in the earliest stages of development. The demonstration of both maternal and zygotic mll transcript expression during embryogenesis as well as mll transcript expression in the adult is consistent with an important role for mll throughout the lifespan of this animal.

The above studies on the temporal expression of zebrafish mll performed by Northern blot and non-quantitative RT-PCR analysis indicate that not only are zebrafish mll transcripts maternally supplied to the embryo, but also that zygotic zebrafish mll is expressed throughout embryogenesis and in the adult. To supplement this data, quantitative RT-PCR analysis was used to study the temporal expression of zebrafish mll mRNA in wild type zebrafish embryos and whole wild type adult. Embryos were pooled and sacrificed at the indicated times. Total RNAs were extracted from the embryos and from a 2-month old whole wild type adult zebrafish using Trizol reagent and the RNAs were treated with DNase. Sense and antisense zebrafish mll specific primers 5′-CAACCCTCAGGAGGAAGATG-3′ (SEQ ID NO: 34) and 5′-CCTGCAGAACAAACCTCTGC-3′ (SEQ ID NO: 35), respectively, from positions 11921-11940 in exon 32 and positions 12086-12067 in exon 34 in the 3′ region of zebrafish mll cDNA corresponding to the SET domain were used to generate to a plasmid subclone for construction of a standard curve (Rutledge et al. (2003) Nuc. Acids Res., 31:e93). The same primers were used for quantitative RT-PCR. The sense and antisense primers to amplify the beta actin (zbactin1) housekeeping gene were 5′-CGAGCAGGAGATGGGAACC-3′ (SEQ ID NO: 36) and 5′-CAACGGAAACGCTCATTGC-3′ (SEQ ID NO: 37), respectively, corresponding to nucleotides 722-740 and 823-805 in exon 4 (GenBank accession no. NM_{—}131031). To generate the standard curves, random hexamer primed first strand cDNA from the whole adult fish was amplified with the zebrafish mll or zbactin1 specific primers and the PCR products were used to generate plasmid subclones containing the relevant zebrafish mll or bactin1 amplicon in the TOPO TA vector (Invitrogen). Each plasmid was linearized with BamHI, the DNA was quantified with a BioPhotometer (Eppendorf) and copy numbers per μL were derived from the number of base pairs in each plasmid, average molecular weight per base pair in double-stranded DNA and the concentration. Standard curves were constructed after performing quantitative real-time PCR on triplicate 10-fold serial dilutions of the linearized plasmids (10⁹ to 10² copies per reaction) using SYBR green and the ABI 7900 HP detection system. The copy number for each reaction was calculated with the SDS software package (ABI). The standard curves had linear ranges between 10² and 10⁸ molecules/μL, and the slopes of both curves were −3.3.

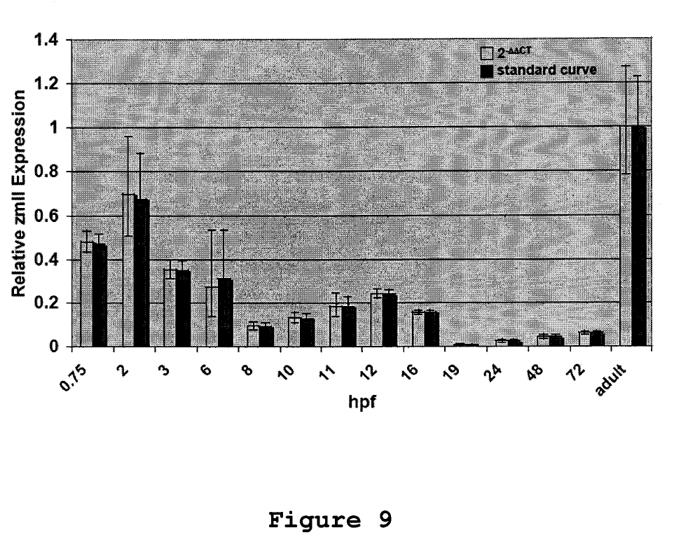

One μg of total RNA from the embryos at the specified timepoints and from the zebrafish adult were used to synthesize random hexamer primed first strand cDNAs using Superscript II reverse transcriptase. A 1 μL aliquot from each cDNA reaction was analyzed in triplicate by quantitative real-time PCR using the same zebrafish mll or zbactin1 primers that were used to generate the standard curves. The mean zebrafish mll copy number was normalized to the mean zbactin1 copy number at each timepoint to determine normalized zebrafish mll copy number from the standard curves. The dark grey bars (FIG. 9) compare the normalized zebrafish mll expression data derived from the standard curves by the absolute quantification method at each timepoint in embryogenesis to the normalized zebrafish mll expression in the adult, with expression values in the embryos shown as fractions of the adult calibrator sample. In addition, the 2^{-ΔΔCT} method (Livak et al. (2001) Methods 25:402-8) was used to analyze the relative changes in zebrafish mll expression as a function of the age of the embryo compared to the adult with expression in the adult calibrator sample set to 1 (light grey bars, FIG. 9). Analysis of the data by the absolute (standard curve) and by the relative (2^{-ΔΔCT}) quantitative methods both gave the same results.

The relative abundance of zebrafish mll mRNA at the different timepoints during embryogenesis was compared to the adult. The quantitative RT-PCR experiment shown in FIG. 9 validates that zebrafish mll mRNA is maternally supplied during the earliest timepoints in the development of the embryo. There also was a peak in zygotic zebrafish mll mRNA expression at 12 hpf in the embryo and the highest relative expression occurred in the zebrafish adult. These experiments illustrate a change in zebrafish mll mRNA expression over time during the life span of the fish.

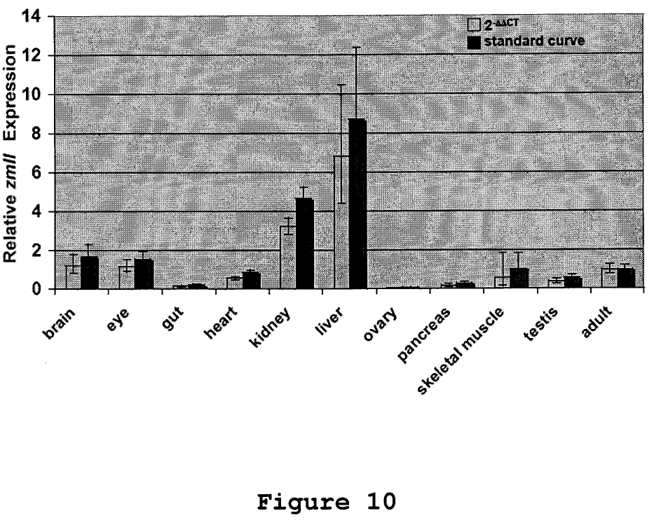

With regard to zebrafish mll mRNA tissue expression, FIG. 10 shows the relative abundance of zebrafish mll mRNA in different tissues compared to the zebrafish mll mRNA expression in the whole adult. Random hexamer primed first strand cDNAs were synthesized from 1 μg of total RNA prepared from the indicated tissues and from a whole wild type adult using Superscript II reverse transcriptase, and a 1 μL aliquot from each cDNA reaction was analyzed in triplicate by quantitative real-time PCR using the same zebrafish mll or zbactin1 primers as described hereinabove. The same standard curves for zebrafish mll and for the zbactin1 housekeeping gene described hereinabove were used to quantify absolute expression of these genes in each tissue and in the whole adult. The relative abundance of zebrafish mll mRNA in the indicated tissues was compared to zebrafish mll mRNA expression in the whole adult by analysis of absolute copy number from the standard curves (dark grey bars) and by analysis of relative gene expression by the 2^{-ΔΔCT} method (light grey bars).

As described hereinabove, the kidney marrow is the site of definitive hematopoiesis in teleosts and normalized zebrafish mll mRNA expression was more abundant in the kidney relative to the whole adult and all other tissues studied with the exception of the liver. Normalized zebrafish mll mRNA expression was also very high in the liver relative to the whole adult and the other tissues. However, zbactin1 expression was very low in the liver, suggesting that the high relative normalized hepatic expression of zebrafish mll may be an overestimate. Indeed, others have recently reported that the hepatic expression of various genes of interest was also overestimated when bactin was used as the internal control (Filbyu et al. (2007) BMC Mol. Biol., 8:10).

### EXAMPLE III

### MLL Deficient Zebrafish

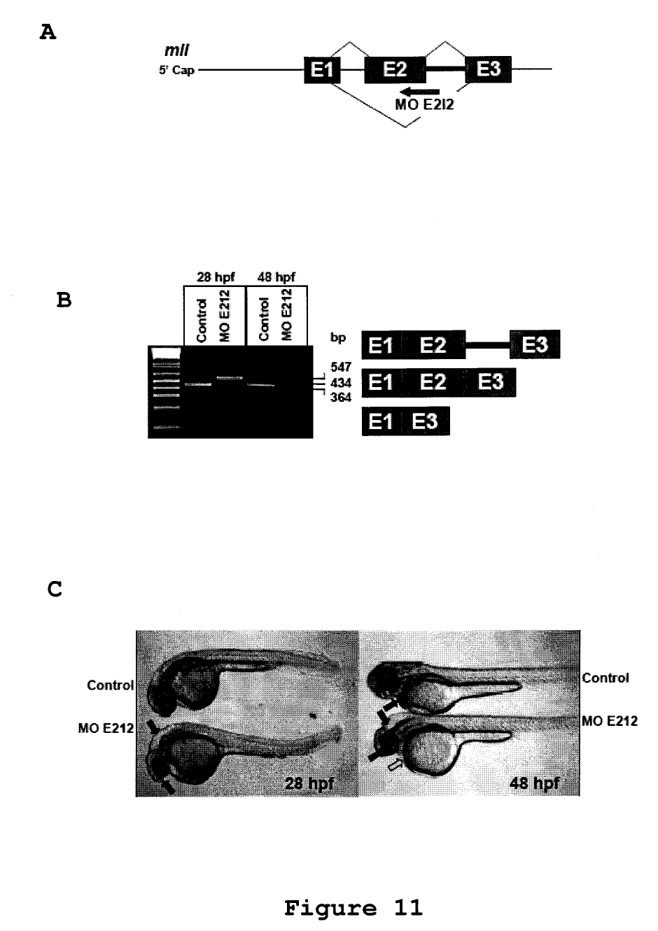

A detailed characterization of the role of wild-type mll in the development of the zebrafish hematopoietic system was also performed. The morpholino knockdown strategy (Paffett-Lugassy et al. (2005) Methods Mol. Med., 105:171-98) was employed to characterize the phenotype of loss of mll and determine whether zebrafish mll depletion is associated with a phenotype resembling that in mammals. Transcriptional processing of mll mRNA was effectively disrupted when newly fertilized embryos were micro-injected at the 1-2 cell stage with a splice-blocking morpholino antisense sequence to the exon 2-intron 2 slice junction (MO E2I2). The construct was obtained from Gene Tools, LLC (Philomath, Oreg.). Target mRNA sequence was 5′-CATAGCCCTGGAAGAACGTCAATAGgtaaacaaattctctaaattattgt-3′ (SEQ ID NO: 38; Exon 2 is capitalized; intron 2 is lower case; underline indicates 25-base sequence encompassed by morpholino). The MO E2I2 sequence was 5′-tagagaatttgtttacCTATTGACG-3′ (SEQ ID NO: 39). The normal transcript splicing is shown by the grey lines in FIG. 11A. The aberrant splicing of exon 1 to exon 3 is shown by the black lines in FIG. 11A and the thick black line in FIG. 11A indicates a second form of aberrant splicing due to failure to splice out intron 2. A 2 mM (16 ng/nl) MO E2I2 stock solution was prepared in dH₂O and diluted in Danieau Solution to the desired concentration.

Wild type male and female adults were bred, fertilized eggs collected, and 100 embryos at the 1-2 cell stage were injected with 16 ng of MO E2I2. Control uninjected embryos (n=100) and injected embryos were raised in petri dishes in E3 embryo medium (Paffett-Lugassy et al. (2005) Methods Mol. Med., 105:171-98) to the desired age. PTU (1-phenyl-2-thiourea) was applied at 24 hpf as described (Herbomel et al. (2005) Methods Mol. Med., 105:199-214) to inhibit melanin synthesis. To harvest embryos for RNA, 20 embryos were collected in 1.5 ml eppendorf tubes, E3/PTU was removed, embryos were anesthetized with tricaine, tricaine was removed, 100% methanol was added and tubes were placed at −20° C. RNA was prepared using RNeasy (Qiagen) and RT-PCR was performed using SuperScript III One Step kit (Invitrogen) with sense and antisense primers 5′-CCGAATTCGAGTCAATGCTT-3′ (SEQ ID NO: 40) and 5′-TTTGGCTGACAGAAGCAGGAG-3′ (SEQ ID NO: 41). Knockdown of properly spliced transcript and production of two aberrant transcripts shown in the schematics of FIG. 11B was confirmed by sequencing the products.

DIC images of representative live embryos were taken using Leica DMRBE microscope with 4× objective and captured using Image Pro. The morphant embryos were viable but exhibited hematopoietic and neuronal defects, small size, and delayed development. Aberrant head protrusion and enlarged hindbrain ventricle were seen at 28 hpf (grey arrows in FIG. 11C). By 48 hpf, erythroid cells are seen in heart/ventral anterior yolk sac of control (filled black arrow in FIG. 11C). In contrast, erythroid cells are barely visible in morphant (unfilled black arrow), and morphant has smaller eyes (arrow) and persistent hindbrain abnormality (arrow) (FIG. 11C).

As indicated in FIG. 11, MO E2I2 inhibited proper splicing and resulted in production of two different aberrantly spliced mll mRNAs and reduction of the normal transcript. Approximately 60% of the 100 embryos injected with MO E2I2 exhibited a phenotype that included hematopoietic and neuronal defects, small size, and delayed development. The morphant embryos were viable but by 28 hpf exhibited an aberrant protrusion at the tip of the head and enlarged hindbrain ventricle. By 48 hpf when erythroid cells were easily visible in the heart and at the ventral yolk sac of control un-injected embryos, substantially less erythroid cells were present at these areas in the morphants. The morphants also had smaller eyes and a prominent hindbrain abnormality.

These findings are of interest because the phenotype of the Mll^{-/-} mouse includes hematopoietic, neuronal, craniofacial and skeletal defects (Yu et al. (1998) PNAS, 95:10632-6; Yu et al. (1995) Nature, 378:505-8), indicating that functional depletion of mll in zebrafish may be associated with similar defects as in mice. That the embryos were small in size is of potential interest also because small size is a feature of Taspase1^{-/-} mice, which results from impaired cell cycle progression when MLL is not cleaved by Taspase 1 (Takeda et al. (2006) Genes Dev., 20:2397-409). The neuronal defect appears to phenocopy that observed in zebrafish following runx1 depletion (Kalev-Zylinska et al. (2002) Development, 129:2015-30).

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An isolated nucleic acid molecule encoding zebrafish MLL, wherein said zebrafish MLL has at least 90% identity with SEQ ID NO: 2.

2. The nucleic acid molecule of claim 1, wherein said zebrafish MLL is SEQ ID NO: 2.

3. The nucleic acid molecule of claim 1 which comprises a nucleotide sequence which has at least 90% identity with SEQ ID NO: 1.

4. The nucleic acid molecule of claim 1 which is SEQ ID NO: 1.

5. An expression vector comprising the nucleic acid molecule of claim 1.

6. An isolated zebrafish MLL protein encoded by the nucleic acid molecule of claim 1.

7. A transgenic zebrafish wherein the expression of zebrafish MLL is reduced compared to wild-type.

8. The transgenic zebrafish of claim 7 which is zebrafish mll null.

9. The transgenic zebrafish of claim 7, wherein said zebrafish comprises an MLL translocation.

10. The transgenic zebrafish of claim 7, wherein the zebrafish comprises an antisense molecule directed to zebrafish mll.

11. The transgenic zebrafish of claim 10, wherein said antisense molecule comprises SEQ ID NO: 39.

12. A method for screening the ability of at least one compound to supplement or replace MLL activity comprising:
a) contacting the zebrafish of claim 7 with said compound; and
b) determining if the compound alters at least one phenotype associated with said zebrafish,
wherein a change in said phenotype to wild-type indicates the ability of said compound to supplement or replace MLL activity.

13. The method of claim 12, wherein said phenotype is reduced erythroid cells in the yolk sac.
